(19) United States
(12) Patent Application Publication
(10) Pub. No.: US 2025/0222221 A1
(43) Pub. Date: Jul. 10, 2025

(54) TRACHEOSTOMY TUBE MONITOR SYSTEMS AND METHODS

(71) Applicant: The University of North Carolina at Chapel Hill, Chapel Hill, NC (US)

(72) Inventors: Jaime Patrick Doody, Chapel Hill, NC (US); Nabil Salih Khan, Chapel Hill, NC (US); Nicole Lewis Wiley, Pittsboro, NC (US); Devin Kerry Hubbard, Chapel Hill, NC (US)

(21) Appl. No.: 18/727,768

(22) PCT Filed: Jan. 10, 2023
(86) PCT No.: PCT/US2023/010487

§ 371 Date: Jul. 10, 2024

Related U.S. Application Data
(60) Provisional application No. 63/297,974, filed on Jan. 10, 2022.

(51) Int. Cl. A61M 16/04; A61F 13/00; A61M 16/00

(52) U.S. Cl. CPC A61M 16/047; A61F 13/00051; A61M 16/0051; A61M 2205/14; A61M 2205/18; A61M 2205/3306; A61M 2210/1032; A61M 2240/00

(57) ABSTRACT

A decannulation detection system includes a dressing, a tube assembly, and a controller. The dressing is applied over a wound or incision site and includes a first electric circuit portion. The tube assembly is positioned in contact with the dressing and includes a second electric circuit portion. When the tube assembly is in contact with the dressing, the first and second electrical circuit portions form a complete electric circuit. The controller is electrically connected to the complete electric circuit and is used to monitor electrical continuity in the complete electric circuit. When the controller detects an electrical discontinuity in the complete electric circuit, the controller triggers an alert regarding the possible occurrence of a decannulation event.

### CROSS-REFERENCE TO RELATED APPLICATION

This application claims priority to U.S. Provisional Patent Application Ser. No. 63/297,974, filed on Jan. 10, 2022, the disclosure of which is incorporated herein by reference in its entirety.

### GOVERNMENT INTEREST

This invention was made with government support under Grant No. TR002489 awarded by the National Institutes of Health. The government has certain rights in the invention.

### BACKGROUND

Accidental tracheostomy dislodgement (decannulation) is the leading cause of death in pediatric patients. Decannulations usually happen when the tracheostomy tube tie is too loose or, otherwise, when the patient coughs or pulls and/or tugs on the tracheostomy tube. Since many pediatric patients with tracheostomy tubes are non-verbal, it is very difficult for the patient to alert a caregiver when a decannulation occurs, especially if the caregiver and the patient are not in the same room. This is further exacerbated because the end of the tracheostomy tube that passes through the stoma may remain hidden from view of the caregiver even during decannulation. When the tracheostomy tube becomes dislodged, the stoma may close before the decannulation of the tracheostomy tube has been noticed, which can lead to asphyxiation of the patient. If the decannulation is noticed before the stoma fully closes, it may be possible for the caregiver to insert the same or, often, a smaller diameter tracheostomy tube to keep the airway open to prevent asphyxiation of the patient until the patient can be transported to a medical site, such as a hospital, for assistance in replacing the smaller tracheostomy tube with the original tracheostomy tube. Given this unmet need and the dire consequences associated with unnoticed decannulation of a tracheostomy tube, a tracheostomy decannulation detection device that can alert caregivers when a decannulation event is occurring or about to occur is urgently needed. When a caregiver is provided with sufficient notice of a decannulation event or an impending event, this would allow an adequate response time for taking remediative actions. Such a decannulation detection device will allow the caregiver to reinsert the tracheostomy tube before the stoma closes or reposition the tracheostomy to prevent total decannulation, decreasing the incidence of death by asphyxiation for pediatric patients with tracheostomy tubes.

### SUMMARY

According to an example embodiment a decannulation detection system is provided herein, the decannulation detection system comprising: a dressing for application over a wound or incision site, the dressing comprising a first electric circuit portion; a tube assembly for positioning in contact with the dressing, the tube assembly comprising a second electric circuit portion; and a controller; wherein, when the tube assembly is in contact with the dressing, the first and second electrical circuit portions form a complete electric circuit; wherein the controller is electrically connected to the complete electric circuit and is configured to monitor electrical continuity in the complete electric circuit; and wherein, when the controller detects an electrical discontinuity in the complete electric circuit, the controller is configured to trigger an alert regarding a possible occurrence of a decannulation event.

In some embodiments of the decannulation detection system, at least a portion of the tube assembly extends at least partially within a wound or incision at the wound or incision site.

In some embodiments of the decannulation detection system, the wound or incision comprises a stoma and the tube assembly comprises a tracheostomy tube assembly.

In some embodiments of the decannulation detection system, the first electrical circuit portion comprises a plurality of discontinuous wires, or foil sheets that are formed in or on an outer surface of the dressing, the outer surface being opposite a surface of the dressing that is against the skin of a patient in which the wound or incision site is formed; the tube assembly comprises a center portion and flanges, the flanges extending from opposite sides of the center portion; the second electrical circuit portion comprises, formed in each of the flanges, at least one electrical contact that protrudes through a surface of a corresponding one of the flanges when the tube assembly contacts the dressing; and when the tube assembly is in contact with the dressing, each of the one or more electrical contacts form an electrical connection between different ones of the plurality of discontinuous wires or foil sheets to form the complete electric circuit.

In some embodiments of the decannulation detection system, the one or more electrical contacts comprise spring-loaded pins.

In some embodiments of the decannulation detection system, when the tube assembly moves away from the dressing during the decannulation event, at least one of the one or more electrical contacts are separated from the dressing to cause the electrical discontinuity in the complete electrical circuit.

In some embodiments of the decannulation detection system, each flange comprises a plurality of electrical contacts spaced apart from each other in an anticipated direction of movement or rotation of the tube assembly away from the dressing during the decannulation event.

In some embodiments of the decannulation detection system, the complete electric circuit is formed only when the tube assembly is in direct contact with the dressing.

According to another example embodiment, a method for detecting a decannulation event is provided, the method comprising: providing a controller; applying a dressing over a wound or incision site, the dressing comprising a first electric circuit portion; positioning a tube assembly adjacent to the dressing, the tube assembly comprising a second electric circuit portion, wherein, when the tube assembly is in contact with the dressing, the first and second electric circuit portions form a complete electric circuit; connecting the controller to the complete electric circuit; monitoring, using the controller, electrical continuity in the complete electric circuit; and triggering, via the controller, an alert regarding a possible occurrence of the decannulation event when the controller detects the electrical discontinuity in the complete electric circuit.

In some embodiments of the method, at least a portion of the tube assembly extends at least partially within a wound or incision at the wound or incision site.

In some embodiments of the method, the wound or incision comprises a stoma and the tube assembly comprises a tracheostomy tube assembly.

In some embodiments of the method, the electrical discontinuity in the complete electric circuit occurs only when the tube assembly has moved at least a predetermined distance away from the dressing, the predetermined distance being correlated with the possible occurrence of the decannulation event.

In some embodiments of the method: the first electrical circuit portion comprises a plurality of discontinuous wires, or foil sheets that are formed in or on an outer surface of the dressing, the outer surface being opposite a surface of the dressing that is against the skin of a patient in which the wound or incision site is formed; the tube assembly comprises a center portion and flanges, the flanges extending from opposite sides of the center portion; the second electrical circuit portion comprises, formed in each of the flanges, at least one electrical contact that protrudes through a surface of a corresponding one of the flanges when the tube assembly contacts the dressing; and when the tube assembly is in contact with the dressing, each of the one or more electrical contacts form an electrical connection between different ones of the plurality of discontinuous wires or foil sheets to form the complete electric circuit.

In some embodiments of the method, the one or more electrical contacts comprise spring-loaded pins.

In some embodiments of the method, when the tube assembly moves away from the dressing during the decannulation event, at least one of the one or more electrical contacts are separated from the dressing to cause the electrical discontinuity in the complete electrical circuit.

In some embodiments of the method, each flange comprises a plurality of electrical contacts spaced apart from each other in an anticipated direction of movement or rotation of the tube assembly away from the dressing during the decannulation event

In some embodiments of the method, the complete electric circuit is formed only when the tube assembly is in direct contact with the dressing.

According to another example embodiment, a decannulation detection system is provided, the decannulation detection device comprising: a tube assembly for positioning in contact with a dressing applied over a wound or incision site or over skin at the wound or incision site, wherein the tube assembly at least partially extends within a wound or incision at the wound or incision site and comprises one or more light sensors; and a controller; wherein the one or more light sensors are positioned on the tube assembly such that light can only be received by the one or more light sensors from a side of the tube assembly that is positioned against the dressing or skin at the wound or incision site; wherein the tube assembly is configured such that, when pressed against the dressing or the skin, no ambient light from outside the tube assembly can be received by the one or more light sensors; wherein, during a decannulation event, the tube assembly is shifted away from the dressing or skin, so as to allow the ambient light to be received by the one or more light sensors; and wherein, when any of the one or more light sensors detects light above a predetermined threshold corresponding to the decannulation event, the controller is configured to trigger an alert regarding a possible occurrence of a decannulation event.

In some embodiments of the decannulation detection system, the wound or incision comprises a stoma and the tube assembly comprises a tracheostomy tube assembly.

In some embodiments of the decannulation detection system, the tube assembly comprises a center portion and flanges, the flanges extending from opposite sides of the center portion; each flange comprises a cavity with a slot formed therein, the slot being formed to extend from the cavity through a surface of the flange that contacts the dressing or the skin; the one or more light sensors comprises a plurality of light sensors, each of the plurality of light sensors being attached to one of the flanges; a photosensor of each light sensor is aligned with the slot of the flange to which such light sensor is attached; and the flanges of the tube assembly comprise a deformable material configured to conform to the surface of the dressing or the skin to block the ambient light from the photosensor of the light sensor when the tube assembly is pressed against the dressing or the skin.

In some embodiments of the decannulation detection system, the deformable material comprises silicone.

In some embodiments of the decannulation detection system, when the tube assembly moves away from the dressing during the decannulation event, one or more of the flanges are separated from the dressing or the skin to allow the ambient light to be incident on the photosensor of a corresponding one of the light sensors.

In some embodiments, the decannulation detection system comprises a light emitter configured to emit a designated wavelength of light, wherein, during the decannulation event, the light sensor is configured to receive light from the light emitter to cause the controller to issue the alert regarding the occurrence of the decannulation event.

In some embodiments of the decannulation detection system, the decannulation detection system is configured to detect the decannulation event in an environment in which no ambient light is present.

In some embodiments of the decannulation detection system, the tube assembly is in direct contact with the dressing or the skin.

According to another example embodiment, a method for detecting a decannulation event is provided, the method comprising: providing a controller; attaching one or more light sensors on the tube assembly; positioning the tube assembly in contact with a dressing applied over a wound or incision site or over skin at the wound or incision site, wherein the tube assembly at least partially extends within a wound or incision at the wound or incision site and comprises one or more light sensors, wherein: the one or more light sensors are positioned on the tube assembly such that light can only be received by the one or more light sensors from a side of the tube assembly that is positioned against the dressing or skin at the wound or incision site; when pressed against the dressing or the skin, no ambient light from outside the tube assembly can be received by the one or more light sensors due to the tube assembly being pressed against the dressing or the skin; and during a decannulation event, the tube assembly is shifted away from the dressing or skin, so as to allow the ambient light to be received by the one or more light sensors; the method further comprising detecting, using the one or more light sensors, light; and triggering, via the controller, an alert regarding a possible occurrence of the decannulation event when any of the one or more light sensors detects light above a predetermined threshold corresponding to the decannulation event.

In some embodiments of the method, the wound or incision comprises a stoma and the tube assembly comprises a tracheostomy tube assembly.

In some embodiments of the method, light is detected by the one or more light sensors only when the tube assembly has moved at least a predetermined distance away from the dressing, the predetermined distance being correlated with the possible occurrence of the decannulation event.

In some embodiments of the method: the tube assembly comprises a center portion and flanges, the flanges extending from opposite sides of the center portion; each flange comprises a cavity with a slot formed therein, the slot being formed to extend from the cavity through a surface of the flange that contacts the dressing or the skin; the one or more light sensors comprises a plurality of light sensors, each of the plurality of light sensors being attached to one of the flanges; a photosensor of each light sensor is aligned with the slot of the flange to which such light sensor is attached; and the flanges of the tube assembly comprise a deformable material configured to conform to the surface of the dressing or the skin to block the ambient light from the photosensor of the light sensor when the tube assembly is pressed against the dressing or the skin.

In some embodiments of the method, the deformable material comprises silicone.

In some embodiments of the method, when the tube assembly moves away from the dressing during the decannulation event, one or more of the flanges are separated from the dressing or the skin to allow the ambient light to be incident on the photosensor of a corresponding one of the light sensors.

In some embodiments, the method comprises emitting, from a light emitter, light having a designated wavelength, wherein, during the decannulation event, the light sensor receives the light having the designated wavelength from the light emitter to cause the controller to issue the alert regarding the occurrence of the decannulation event.

In some embodiments of the method, the decannulation detection system can detect the decannulation event in an environment in which no ambient light is present.

In some embodiments of the method, the tube assembly is in direct contact with the dressing or the skin.

According to another example embodiment, a decannulation detection system is provided, the decannulation detection device comprising: a dressing for application over a wound or incision site, the dressing comprising one or more first capacitive plates; a tube assembly for positioning in contact with the dressing and comprising one or more second capacitive plates; an electrical circuit that is connected to the one or more first capacitive plates and the one or more second capacitive plates; and a controller that is configured to detect and monitor a capacitance between the one or more first capacitive plates and the one or more second capacitive plates, wherein the capacitance varies based on a distance between the one or more first capacitive plates and the one or more second capacitive plates; wherein, when the controller detects that the capacitance between the one or more first capacitive plates and the one or more second capacitive plates is not within a predetermined range of capacitance values or is above or below a threshold capacitance value, the controller is configured to trigger an alert regarding a possible occurrence of a decannulation event.

In some embodiments of the decannulation detection system, at least a portion of the tube assembly extends at least partially within a wound or incision at the wound or incision site.

In some embodiments of the decannulation detection system, the wound or incision comprises a stoma and the tube assembly comprises a tracheostomy tube assembly.

In some embodiments, the decannulation detection system comprises an insulator positioned directly between each of one or more first capacitive plates and/or the one or more second capacitive plates to prevent direct contact between one or more first capacitive plates and the one or more second capacitive plates.

In some embodiments of the decannulation detection system, when the tube assembly moves away from the dressing during the decannulation event, the one or more first capacitive plates and the one or more second capacitive plates are moved away from each other by a corresponding distance, which causes a change in the capacitance measured between the one or more first capacitive plates and the one or more second capacitive plates.

In some embodiments of the decannulation detection system, the tube assembly comprises a center portion and flanges, the flanges extending from opposite sides of the center portion.

In some embodiments of the decannulation detection system, each flange comprises at least one of the one or more first capacitive plates, such that the tube assembly comprises a plurality of first capacitive plates and the dressing comprises a same quantity of the one or more second capacitive plates as a total quantity of plurality of first capacitive plates.

According to another example embodiment, a method for detecting a decannulation event is provided, the method comprising: applying a dressing over a wound or incision site, the dressing comprising one or more first capacitive plates; positioning the tube assembly in contact with the dressing, the tube assembly comprising one or more second capacitive plates; providing an electrical circuit that is connected to the one or more first capacitive plates and the one or more second capacitive plates; detecting, using a controller, a capacitance between the one or more first capacitive plates and the one or more second capacitive plates, wherein the capacitance varies based on a distance between the one or more first capacitive plates and the one or more second capacitive plates; monitoring the capacitance; and triggering, using the controller, an alert regarding a possible occurrence of the decannulation event when the controller detects that the capacitance between the one or more first capacitive plates and the one or more second capacitive plates is not within a predetermined range of capacitance values or is above or below a threshold capacitance value.

In some embodiments of the method, at least a portion of the tube assembly extends at least partially within a wound or incision at the wound or incision site.

In some embodiments of the method, the wound or incision comprises a stoma and the tube assembly comprises a tracheostomy tube assembly.

In some embodiments, the method comprises positioning an insulator directly between each of one or more first capacitive plates and/or the one or more second capacitive plates to prevent direct contact between one or more first capacitive plates and the one or more second capacitive plates.

In some embodiments of the method, when the tube assembly moves away from the dressing during the decannulation event, the one or more first capacitive plates and the one or more second capacitive plates are moved away from each other by a corresponding distance, which causes a change in the capacitance measured between the one or more first capacitive plates and the one or more second capacitive plates.

In some embodiments of the method, the tube assembly comprises a center portion and flanges, the flanges extending from opposite sides of the center portion.

In some embodiments of the method, each flange comprises at least one of the one or more first capacitive plates, such that the tube assembly comprises a plurality of first capacitive plates and the dressing comprises a same quantity of the one or more second capacitive plates as a total quantity of plurality of first capacitive plates.

According to another example embodiment, a decannulation detection system is provided, the decannulation detection device comprising: a dressing for application over a wound or incision site, the dressing comprising one or more first inductors; a tube assembly for positioning in contact with the dressing and comprising one or more second inductors; and an electric circuit that is connected to the one or more first inductors and the one or more second inductors; and a controller that is configured to detect and monitor an inductance, optionally a mutual inductance, between the one or more first inductors and the one or more second inductors, wherein the inductance varies based on a distance between the one or more first inductors and the one or more second inductors; wherein, when the controller detects that the inductance between the one or more first inductors and the one or more second inductors is not within a predetermined range of inductance values or is above or below a threshold inductance value, the controller is configured to trigger an alert regarding a possible occurrence of a decannulation event.

In some embodiments of the decannulation detection system, at least a portion of the tube assembly extends at least partially within a wound or incision at the wound or incision site.

In some embodiments of the decannulation detection system, the wound or incision comprises a stoma and the tube assembly comprises a tracheostomy tube assembly.

In some embodiments of the decannulation detection system, the one or more first inductors each comprise coils of a wire in the electric circuit; and/or the one or more second inductors each comprise coils of a wire in the electric circuit.

In some embodiments of the decannulation detection system, when the tube assembly moves away from the dressing during the decannulation event, the one or more first inductors and the one or more second inductors are moved away from each other by a corresponding distance, which causes a change in the inductance measured between the one or more first inductors and the one or more second inductors.

In some embodiments of the decannulation detection system, the tube assembly comprises a center portion and flanges, the flanges extending from opposite sides of the center portion.

In some embodiments of the decannulation detection system, each flange comprises at least one of the one or more first inductors, such that the tube assembly comprises a plurality of first inductors and the dressing comprises a same quantity of the one or more second inductors as a total quantity of plurality of first inductors.

According to another example embodiment, a method for detecting a decannulation event is provided, the method comprising: applying a dressing over a wound or incision site, the dressing comprising one or more first inductors; positioning the tube assembly in contact with the dressing, the tube assembly comprising one or more second inductors; providing an electric circuit that is connected to the one or more first inductors and the one or more second inductors; detecting, using a controller, an inductance, optionally a mutual inductance, between the one or more first inductors and the one or more second inductors, wherein the inductance varies based on a distance between the one or more first inductors and the one or more second inductors; monitoring the inductance; and triggering, using the controller, an alert regarding a possible occurrence of the decannulation event when the controller detects that the inductance between the one or more first inductors and the one or more second inductors is not within a predetermined range of inductance values or is above or below a threshold inductance value.

In some embodiments of the method, at least a portion of the tube assembly extends at least partially within a wound or incision at the wound or incision site.

In some embodiments of the method, the wound or incision comprises a stoma and the tube assembly comprises a tracheostomy tube assembly.

In some embodiments of the method, the one or more first inductors each comprise coils of a wire in the electric circuit; and/or the one or more second inductors each comprise coils of a wire in the electric circuit.

In some embodiments of the method, when the tube assembly moves away from the dressing during the decannulation event, the one or more first inductors and the one or more second inductors are moved away from each other by a corresponding distance, which causes a change in the inductance measured between the one or more first inductors and the one or more second inductors.

In some embodiments of the method, the tube assembly comprises a center portion and flanges, the flanges extending from opposite sides of the center portion.

In some embodiments of the method, each flange comprises at least one of the one or more first inductors, such that the tube assembly comprises a plurality of first inductors and the dressing comprises a same quantity of the one or more second inductors as a total quantity of plurality of first inductors.

### BRIEF DESCRIPTION OF THE DRAWINGS

FIG. 1 is a sketch of an example embodiment of a tracheostomy tube assembly configured for detection of a decannulation event.

FIG. 2 is a sketch of an example embodiment of an electrical circuit for detection of a decannulation event.

FIG. 3 is a sketch of an example embodiment of an electrically conductive dressing for use with the tracheostomy tube assembly of FIG. 1 in detecting a decannulation event.

FIG. 4 shows various examples of types of known tracheostomy tubes.

FIG. 5 is an isometric view of an example embodiment of a tracheostomy tube assembly configured for detection of a decannulation event by monitoring electrical continuity through a circuit.

FIG. 6 is a front view of the tracheostomy tube assembly of FIG. 5.

FIG. 7 is a front view of a contact switch sensor housing for attachment to the tracheostomy tube assembly shown in FIGS. 5 and 6.

FIG. 8 is a rear view of the contact switch sensor housing shown in FIG. 7.

FIG. 9 is a front view of a contact switch sensor assembly, which includes the contact switch sensor housing shown in FIGS. 7 and 8.

FIG. 10 is a rear view of the contact switch sensor assembly shown in FIG. 9.

FIG. 11 is a cross-sectional view of the contact switch sensor assembly shown in FIGS. 9 and 10, along sectional plane 11-11.

FIG. 12 is a cross-sectional view of the contact switch sensor assembly shown in FIGS. 9 and 10, along sectional plane 12-12.

FIG. 13 is an alternate example embodiment of the tracheostomy tube assembly shown in FIGS. 5 and 6.

FIG. 14 is an example electrical schematic for monitoring electrical continuity in a decannulation detection system.

FIG. 15 is another example electrical schematic for monitoring electrical continuity in a decannulation detection system.

FIG. 16 is another example electrical schematic for monitoring electrical continuity in a decannulation detection system.

FIG. 17 shows the tracheostomy tube assembly shown in FIG. 13 attached about the neck of a simulated pediatric patient as part of a decannulation detection system.

FIG. 18 is an isometric view of an example embodiment of a tracheostomy tube assembly configured for detection of a decannulation event using light detection.

FIG. 19 is a front view of the tracheostomy tube assembly of FIG. 18.

FIG. 20 is a front view of a light sensor housing for attachment to the tracheostomy tube assembly shown in FIGS. 18 and 19.

FIG. 21 is a rear view of the light sensor housing shown in FIG. 20.

FIG. 22 is a front view of a light sensor assembly, which includes the light sensor housing shown in FIGS. 20 and 21.

FIG. 23 is a rear view of the light sensor assembly shown in FIG. 22.

FIG. 24 is a cross-sectional view of the light sensor housing shown in FIG. 20, along sectional plane 24-24.

FIG. 25 is a cross-sectional view of the ambient light sensor assembly shown in FIG. 22, along sectional plane 25-25.

FIG. 26 is an electrical schematic of an ambient light sensor circuit for monitoring ambient light in a decannulation detection system.

FIG. 27 is a block diagram of an example embodiment of software components in a decannulation detection system.

FIG. 28 is a flow chart for example operation of a decannulation detection system.

FIG. 29 is a partially exploded isometric view of an example embodiment of a decannulation detection system comprising for detection of a decannulation event using capacitance.

FIG. 30 is a partially exploded perspective view of the decannulation detection system of FIG. 29.

FIG. 31 is an electrical schematic of a capacitance monitoring circuit for use in the example embodiment of the decannulation detection system of FIGS. 29 and 30.

FIG. 32 is a partially exploded isometric view of an example embodiment of a decannulation detection system for detection of a decannulation event using inductance.

FIG. 33 is a perspective rear isometric view of the tracheostomy tube assembly of the decannulation detection system of FIG. 32.

FIG. 34 is a partially exploded side plan view of the decannulation detection system of FIG. 32.

FIG. 35 is a front plan view of the dressing of the decannulation detection system of FIG. 32.

FIG. 36 is an electrical schematic of an inductance monitoring circuit for use in a decannulation detection system, an example of which is shown in FIGS. 32-35.

### DETAILED DESCRIPTION

The presently disclosed subject matter now will be described more fully hereinafter, in which some, but not all embodiments of the presently disclosed subject matter are described. Indeed, the presently disclosed subject matter can be embodied in many different forms and should not be construed as limited to the embodiments set forth herein; rather, these embodiments are provided so that this disclosure will satisfy applicable legal requirements.

To meet the aforementioned challenges and requirements, disclosed herein are example systems and methods for using a tracheostomy tube assembly to detect the occurrence of a movement of a tube assembly (e.g., a tracheostomy tube assembly, but not being limited thereto) to and/or beyond a predetermined amount, or degree, of movement. Movement of the tube assembly to and/or beyond the predetermined amount of movement is indicative of the occurrence of a decannulation event, in which the tracheostomy tube is removed fully or partially from the stoma, that may otherwise go unnoticed in a sufficient amount of time for a caregiver to take remediative actions.

Unless defined otherwise, all technical and scientific terms used herein have the same meaning as commonly understood to one having ordinary skill in the art to which the presently disclosed subject matter belongs. Although any methods, devices, and materials similar or equivalent to those described herein can be used in the practice or testing of the presently disclosed subject matter, representative methods, devices, and materials are now described.

Following long-standing patent law convention, the terms “a”, “an”, and “the” refer to “one or more” when used in this application, including the claims. Thus, for example, reference to “a capacitive plate” can include a plurality of such capacitive plates, and so forth.

Unless otherwise indicated, all numbers expressing quantities of length, diameter, width, and so forth used in the specification and claims are to be understood as being modified in all instances by the terms “about” or “approximately”. Accordingly, unless indicated to the contrary, the numerical parameters set forth in this specification and attached claims are approximations that can vary depending upon the desired properties sought to be obtained by the presently disclosed subject matter.

As used herein, the terms “about” and “approximately,” when referring to a value or to a length, width, diameter, temperature, time, volume, concentration, percentage, etc., is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate for the disclosed apparatuses and devices.

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole. As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, the term “and/or” when used in the context of a listing of entities, refers to the entities being present singly or in combination. Thus, for example, the phrase “A, B, C, and/or D” includes A, B, C, and D individually, but also includes any and all combinations and sub-combinations of A, B, C, and D.

The term “subject”, “individual”, and “patient” are used interchangeably herein, and refer to an animal, especially a mammal, for example a human, to whom treatment or monitoring, with a device or system as described herein, is provided. The term “mammal” is intended to encompass a singular “mammal” and plural “mammals,” and includes, but is not limited: to humans, primates such as apes, monkeys, orangutans, and chimpanzees; canids such as dogs and wolves; felids such as cats, lions, and tigers; equids such as horses, donkeys, and zebras, food animals such as cows, pigs, and sheep; ungulates such as deer and giraffes; rodents such as mice, rats, hamsters and guinea pigs; and bears.

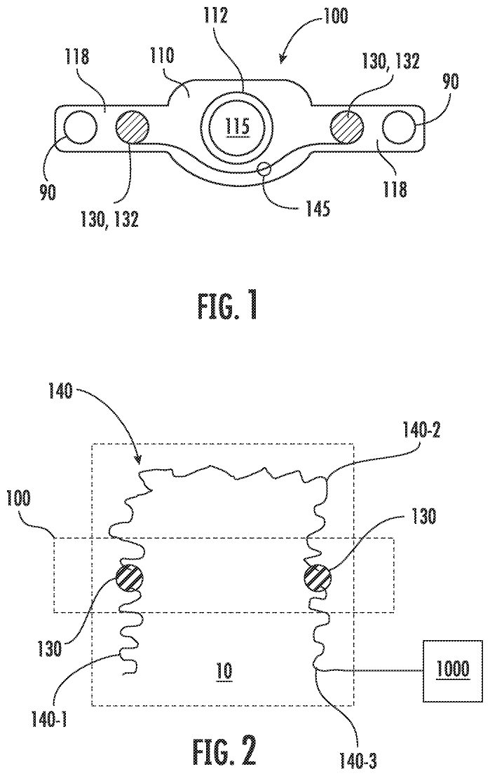

FIG. 1 is an example of a tracheostomy tube assembly, generally designated 100, configured to form, in conjunction with the dressing 10 shown in FIG. 2, an electrical circuit, generally designated 140, for monitoring a position of the tracheostomy tube assembly 100 relative to the dressing 10, detecting a decannulation event, and alerting a caregiver to the occurrence of such a decannulation event. As shown in FIG. 1, the tracheostomy tube assembly 100 comprises a main body 110, which in turn comprises a generally annularly-shaped central portion 112 and flanges 118, or flanged portions, that are formed so as to extend away from each other on opposite sides of the central portion 112. The central portion 112 comprises a tracheostomy tube hole 115 formed therethrough (e.g., entirely therethrough) for passage of a tracheostomy tube through the tracheostomy tube hole 115. The tracheostomy tube hole 115 may be formed at any suitable position on the central portion 110, including the coaxial position as shown in FIG. 1. The outer perimeter of the central portion 110 can be of any suitable shape, including any suitable polygonal or irregular shape. The flanges 118 extend radially outwardly from the tracheostomy tube hole 115 formed in the central portion 112. Each flange 118 comprises a contact hole 132 and a tie hole 90 that are both formed through the thickness of (e.g., through the entire thickness of) the flange 118 and that extend, when the central portion 112 and the flanges 118 are in the same plane (e.g., in a flat position), in substantially the same direction as the tracheostomy tube hole 115; thus, in the view shown in FIG. 1, the longitudinal axis of each of the contact hole 132, the tie hole 90, and the tracheostomy tube hole 115 is parallel with each other, respectively. The contact hole 132 is positioned radially inwardly towards the tracheostomy tube hole 115, relative to the tie hole 90. The tracheostomy tube assembly 100 comprises, for each contact hole 132, an electrical contact 130 that is embedded therein (e.g., in a press-fit, or interference fit, manner). The tie holes 90 are each configured to receive and have secured thereto a tracheostomy tube tie for securing the tracheostomy tube assembly 100 in a substantially fixed position over the stoma. As shown in FIG. 1, the tracheostomy tube assembly 100 comprises an interconnecting wire 145 that is embedded within, or otherwise attached to, the main body 110 of the tracheostomy tube assembly 100, such that the wire 145 electrically connects the electrical contact 130 in one flange 118 (e.g., the left flange 118, as shown in FIG. 1) to the electrical contact 130 in the other flange 118 (e.g., the right flange 118, as shown in FIG. 1).

As shown in FIG. 2, the dressing 10, which covers the stoma and is substantially rigidly connected (e.g., via an adhesive, such as tape) to the surface of the skin of the patient, comprises a circuit, generally designated 140, formed in or on the surface of the dressing 10. In FIG. 2, the outer edges of the dressing 10 are shown in broken line. Similarly, the tracheostomy tube assembly 100 is schematically shown in broken line, with the electrical contacts 130 being positioned to provide electrical continuity to the three wires 140-1, 140-2, 140-3 that form the circuit 140 in the dressing 10. Thus, the first wire 140-1 is electrically connected to the second wire 140-2 by a first electrical contact 130 (e.g., on the left side of FIG. 2) and the second wire 140-2 is electrically connected to the third wire 140-3 by a second electrical contact 130 (e.g., on the right side of FIG. 2). The circuit 140 is connected to a controller 1000 that is configured to trigger an alert (e.g., an auditory, haptic, visual signal, including display of a text-based alert message on an electronic device, which can be, for example and without limitation, a mobile phone of a caregiver) upon a loss of electrical continuity between the electrical contacts 130 of the tracheostomy tube assembly 100 and the wires 140-1, 140-2, 140-3 that form the circuit 140 of the dressing 10, which may occur, for example, during a decannulation event. In some embodiments, the controller 1000 comprises an alarm. Thus, the loss of electrical continuity within the circuit 140 due to a disruption of electrical continuity between any of the wires 140-1, 140-2, 140-3 that form the circuit 140 and the electrical contacts 130, such as may occur due to movement of the tracheostomy tube assembly 100 away from (e.g., in the direction perpendicular to the plane in which FIG. 2 is shown) the dressing 10 is able to alert a caregiver as to the possible occurrence of a decannulation event.

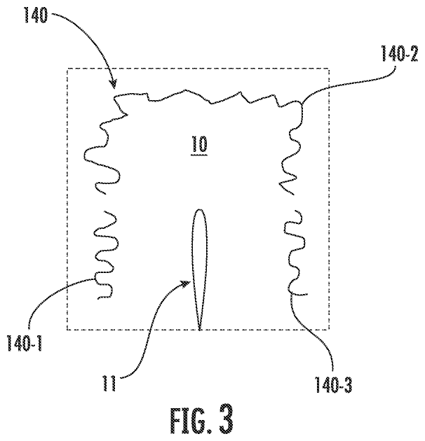

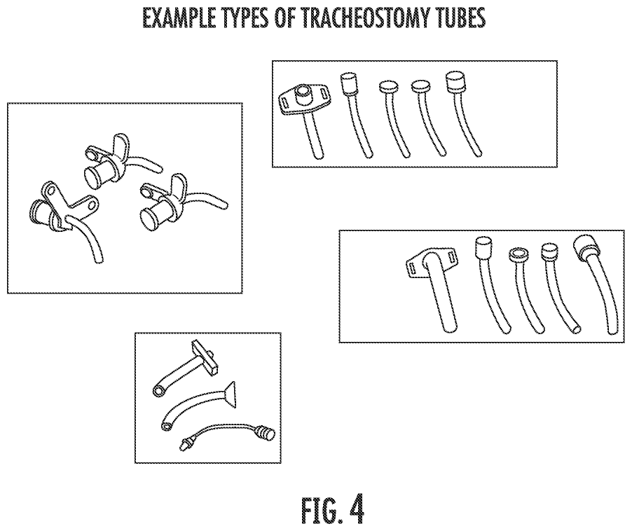

FIG. 3 is an alternate embodiment for the dressing 10 in which the wires 140-1, 140-2, 140-3 that form the circuit 140 are shown embedded within (e.g., partially or entirely) the dressing 10. The dressing 10 may have, for example to allow for ease of positioning of the dressing 10 around a stoma a slit 11 formed therein, extending from an edge of the dressing 10 in an inwards manner, such as towards a center of the dressing 10. FIG. 4 shows a nonlimiting set of various examples of existing tracheostomy tubes that can be held securely in place by any of the tracheostomy tube assemblies disclosed herein, as well as variants of such tracheostomy tube assemblies that would be obvious to a person having ordinary skill in the art.

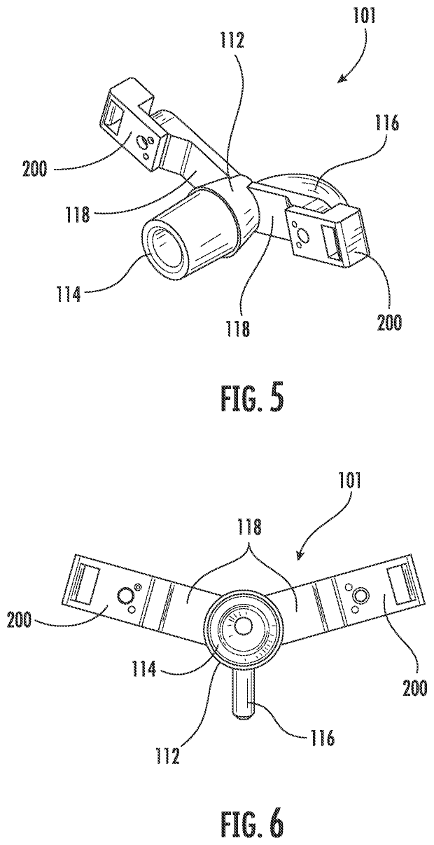

FIGS. 5 and 6 show various aspects of an example embodiment of a tracheostomy tube assembly, generally designated 101, for use in a decannulation detection system that monitors electrical continuity between the tracheostomy tube assembly 101 and the dressing (e.g., 10, FIG. 2 or 3) attached to the skin of the patient around/over the stoma. The features of the tracheostomy tube assembly 101 are generally similar to the features of the tracheostomy tube assembly 100 and features that are not specifically described herein as being different with respect to the tracheostomy tube assembly 101 are substantially identical to the same features of the tracheostomy tube assembly 100. Differences in illustration in the figures of features of the tracheostomy tube assembly 100 and the tracheostomy tube assembly 101 do not necessarily indicate a difference in structure or function of such features unless such difference(s) are specifically described herein with respect to the tracheostomy tube assembly 101. In this example embodiment of the tracheostomy tube assembly 101, the electrical contact 130 is not inserted directly into the flange 118, but is instead contained within one of the sensor housings 200.

As shown in FIGS. 5 and 6, the tracheostomy tube assembly 101 comprises sensor housings 200 that are attached (e.g., rigidly, removably, permanently, or in any suitable manner) to a conventional tracheostomy tube holder, thereby allowing for a retrofit application of known tracheostomy tube holders as part of such a decannulation detection system. The tracheostomy tube holder shown in FIGS. 5 and 6 is a BIVONA® Pediatric Tracheostomy Tube. Just as was described generally with regard to FIG. 1, the tracheostomy tube body comprises a central portion 112, with a tracheostomy tube hole formed therethrough and with the tracheostomy tube 116 attached rigidly to extend from a rear side of the central portion 112 to extend through the stoma. The central portion 112 further comprises, on a front side thereof, a raised portion 114 to aid in preventing unintended blockage of the tracheostomy tube hole that may otherwise lead to a hypoxic condition for the patient. The raised portion 114 and the tracheostomy tube 114 may be rigidly connected to each other, so as to be inserted through the tracheostomy tube hole formed through the central portion 112 in a unitary manner. The tracheostomy tube body also comprises, attached on opposing sides of the central portion 112, flanges 118 that extend radially outwardly from the tracheostomy tube hole formed in the central portion 112. As used herein, the term “radially” can mean, for example, in a direction coaxial with a radial line passing through the longitudinal axis of the tracheostomy tube hole.

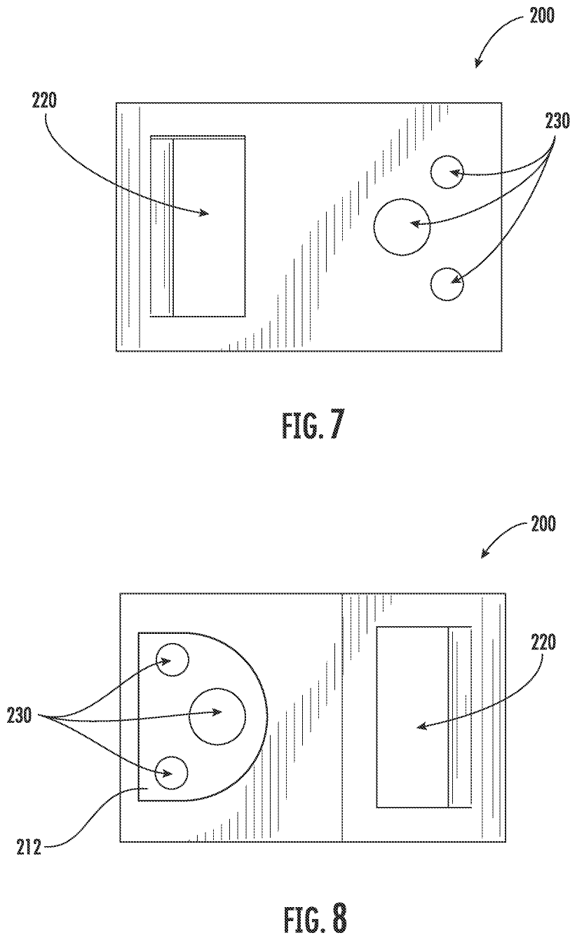

Each flange 118 comprises a tie hole (e.g., 90, see FIG. 1) formed through (e.g., entirely through) the thickness of the flange 118. In some embodiments, only the central portion 112 and the flanges 118 of the tracheostomy tube assembly 101 are formed as a unitary, or monolithic structure (e.g., in a single piece). In some embodiments, only the central portion 112, the tracheostomy tube 116, the raised portion 114, and the flanges 118 of the tracheostomy tube assembly 101 are formed as a unitary, or monolithic structure (e.g., in a single piece). In order for such a known tracheostomy tube body to be used as part of a decannulation detection system, a contact switch sensor housing 200 is attached (e.g., in a press-fit, or interference fit, manner, or via any other suitably rigid attachment type) by insertion through one of the tie holes formed through the flange, thereby forming a quasi-extension of the flange 118. Aspects of the contact switch sensor housing 200 are further illustrated in FIGS. 7-12.

FIGS. 7 and 8 are respective front and rear views of an example embodiment of a contact switch sensor housing, generally designated 200. On one end of the contact switch sensor housing 200 a protrusion 212 is formed, the protrusion 212 having a substantially similar cross-sectional shape to that of the tie holder formed in the flange 118 of the conventional tracheostomy tube holder, such that the protrusion 212 can be inserted within the tie holder of the conventional tracheostomy tube holder and be secured therein (e.g., via an interference fit or other suitably rigid mechanical attachment type). There are a plurality of (e.g., three) holes, generally designated 230 that are formed through the protrusion 212, these holes 230 being formed to extend through the entire thickness of the protrusion 212. These holes 230 are shaped to receive an electrical contact 130 through at least a portion of, or all of, a corresponding one of the holes 230. In the example embodiment shown, the electrical contacts 130 each have a so-called “pogo-pin” (e.g., a Mill-Max spring-loaded electrically conductive pin) design that is configured to maintain a closed electrical circuit when such pin of the electrical contact 130 is in contact with the tracheostomy dressing (e.g., Mepilex, see 10, FIGS. 2 and 3), which comprises a metallic material, such as wire or foil. Thus, in the example embodiment shown in FIGS. 5-12, the electrical contacts 130 are spring-loaded contacts and are press-fit within one of the holes 230 until flush with the outer surface of the flange 118 of the tracheostomy tube body.

The electrical contact 130 in the form of a spring-loaded pin provides the ability to accommodate varying anatomy and small movement (e.g., breathing, etc.) without resulting in false alerts for the occurrence of a decannulation event. If the tracheostomy tube 116 becomes partially or completely dislodged, the spring pin electrical contact 130 will break contact with (e.g., become electrically discontinuous with) the metallic material of the dressing and the detection circuit (e.g., 140, see FIGS. 2 and 3) is configured to issue an alarm to notify caregivers of the occurrence of a possible decannulation event. There are multiple configurations for electrical contacts 130 and detection circuits 140 that can be used to provide redundancy. The electrical contacts 130 are not necessarily limited to such spring-loaded embodiments and can include without limitation any suitable electrically conductive element. In some embodiments, a plate may extend, on the surface of the contact switch sensor housing 200 that is adjacent to the dressing 10 when the tracheostomy tube assembly 101 is installed on a patient, between electrical contacts 130 (e.g., pins) held within the holes 230 of the contact switch sensor housing 200, in the manner of a conductive plate, with the engagement of the pin-shaped electrical contacts 130 within the holes 20 resisting movement of the plate in any direction. On an end of the contact switch sensor housing 200, opposite from where the protrusion 212 is formed, a slot 220 is provided, the slot 220 being configured in the manner of a tracheostomy tube tie for securing the tracheostomy tube assembly 101 in a substantially fixed position over the stoma.

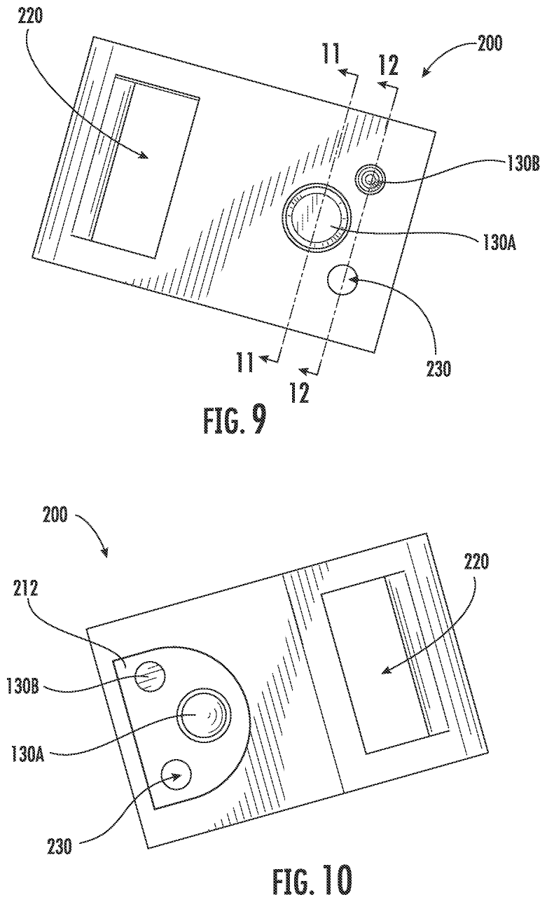

As shown in FIGS. 9 and 10, a plurality of electrical contacts 130 (e.g., Mill-Max spring-loaded contact pins) are provided, each electrical contact 130 being inserted through a corresponding one of the holes 230 that are formed in the protrusion 212 of the contact switch sensor housing 200. In the example embodiment shown, up to three electrical contacts 130 can be installed per contact switch sensor housing 200 to allow for a plurality of different electrical circuit detection designs or configurations.

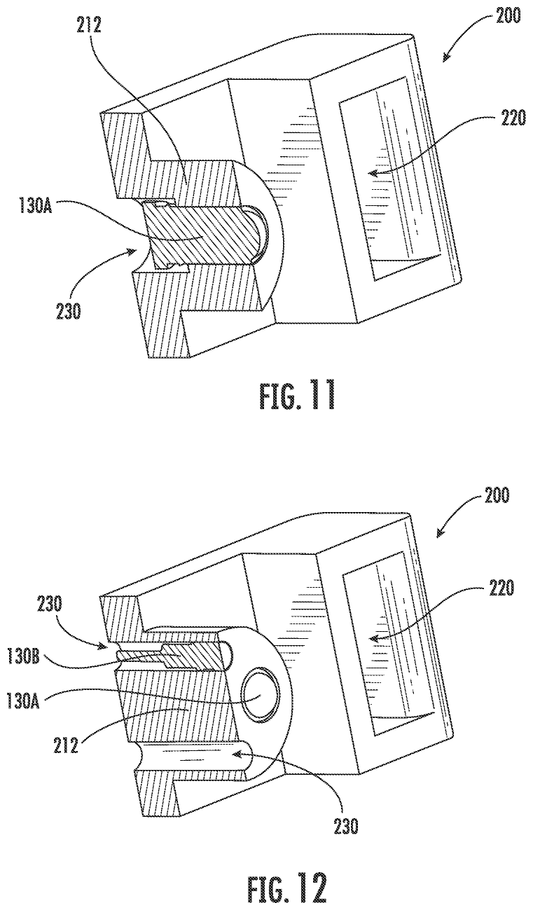

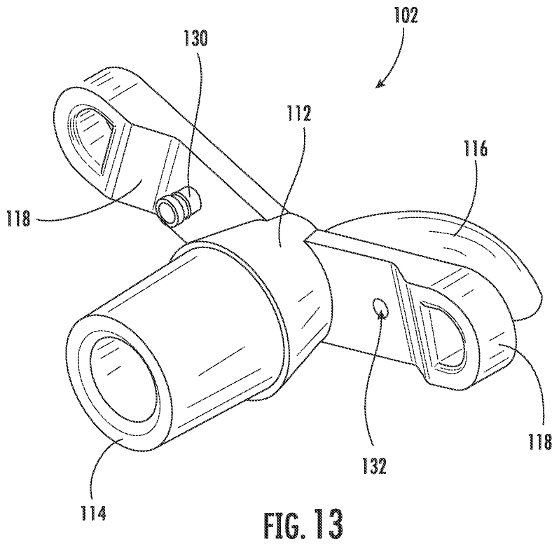

FIGS. 11 and 12 are respective cross-sectional views of one of the contact switch sensor housings 200 to show various aspects of the electrical contacts 130 shown in FIGS. 9 and 10. FIG. 11 is a cross-sectional view taken along cut-plane 11-11 in FIG. 9. As shown in FIG. 11, the electrical contact 130A is a Mill-Max 0945-0-15-20-09-14-11-0 Spring Loaded Contact Pin, which is installed within one of the holes 230 formed through the protrusion 212 of the contact switch sensor housing 200. The electrical contact 130A shown in FIG. 11 has a rounded, or domed (e.g., in a hemispherical manner), shape that protrudes from the hole 230 in which such electrical contact 130A is retained. The electrical contact 130A shown in FIG. 11 can be of any suitable type. FIG. 12 is a cross-sectional view taken along cut-plane 12-12 in FIG. 9. As shown in FIG. 12, one of the other holes 230 formed through the protrusion 212 of the contact switch sensor housing 200 is populated with (e.g., has inserted therein) an electrical contact 130B, which is a different type of electrical contact from electrical contact 130A. In the example embodiment disclosed herein, the electrical contact 130B is a Mill-Max 1951-0-00-15-00-00-03-0 Target Contact with Flat Face. In this example embodiment, the other hole 230 is shown being left vacant, with no electrical contact 130 installed therein. The use of multiple positionally staggered electrical contacts 130 can advantageously be used to detect a partial separation of (e.g., via a relative rotational movement therebetween) the tracheostomy tube assembly 101 and the dressing 10.

An alternate embodiment of a tracheostomy tube assembly, generally designated 102, which is configured to detect a decannulation event via monitoring electrical continuity, is shown in FIG. 13. The tracheostomy tube assembly shown in FIG. 13 is structurally substantially identical to the conventional tracheostomy tube holder shown in FIGS. 5 and 6, except as described as being different therefrom herein. In the example embodiment of FIG. 13, the tracheostomy tube assembly 102 omits the contact switch sensor housing 200 shown in FIGS. 5 and 6. Instead of the contact switch sensor housing 200, the tracheostomy tube assembly 102 has one or more (e.g., a plurality of) holes 132 formed in the flanges 118. The holes 132 are configured for the insertion of electrical contacts 130 therein. As shown, the holes 132 are formed in the flanges 118 of the tracheostomy tube assembly 102 directly. While a single hole 132 is shown being formed in each of the flanges 118 in FIG. 13, any holes 132 may be provided in the flanges 118 in any quantity, configuration, size (e.g., diameter), and position based on the electrical circuit being formed for detection of a decannulation event. Provision of holes 132 (and electrical contacts 130) along the length of (e.g., in the direction radially away from the central portion 112) the flange 118 can be used to detect separation of a portion of the tracheostomy tube assembly 102 away from the dressing that may not otherwise be detected by a single electrical contact 130 positioned within a single hole 132 for each flange 118, as is the configuration shown in FIG. 13.

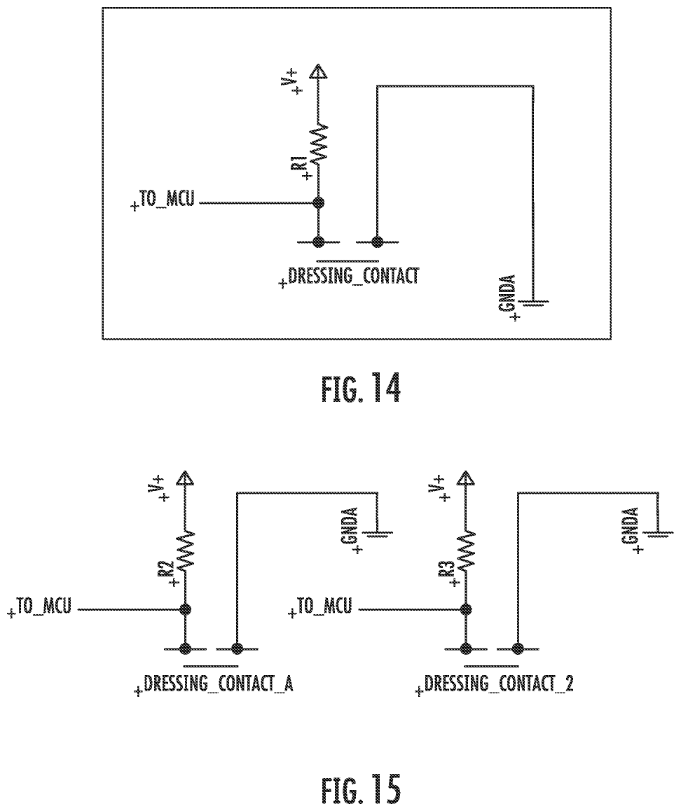

FIG. 14 shows an example embodiment of an electrical circuit that can be formed between the dressing (e.g., 10, FIGS. 2 and 3) and the tracheostomy tube assemblies 101, 102 of any of FIGS. 5-13. As shown in FIG. 14, the circuit shown therein is a push button circuit to implement the contact switch sensor. Any suitably electrically conductive material (e.g., copper wire or foil) can be secured on, in, and/or to the top surface (e.g., the surface facing away from the skin of the patient) of the dressing 10 to provide a conductive path between the electrical contacts 130, whether installed in the contact switch sensor housings 200 shown in FIGS. 5-12 or in the holes shown in FIGS. 13. In the example embodiments disclosed herein, the electrical contacts 130, in the form of Mill-Max 0945-0-15-20-09-14-11-0 Spring Loaded Contact Pins, were populated on both contact switch sensor housings (see, e.g., 200, FIG. 11) to form the electrical circuit shown in FIG. 14. Port 0 Pin 5 was configured as an External IRQ 10 to detect Open/Shorted Circuit conditions on the sensors.

FIG. 15 shows another example embodiment of an electrical circuit that can be formed between the dressing (e.g., 10, FIGS. 2 and 3) and the tracheostomy tube assemblies 101, 102 of any of FIGS. 5-13. As shown in FIG. 15, the electrical circuit allows for granular independent detection of electrical continuity between the electrical contacts 130 on the opposite sides of the central portion 112, whether retained in the contact switch sensor housings 200 disclosed in FIGS. 5-12 or in the holes 132 disclosed in

FIG. 13. In the configuration for the electrical circuit shown in FIG. 15, there are two (2) electrical contacts 130 (e.g., Mill-Max 1951-0-00-15-00-00-03-0 Target Contacts) that are populated in two of the holes 230 formed in the contact switch sensor housings 200 (see, e.g., FIGS. 7-12). The same electrical configuration can be provided by the formation of at least two holes 132 in each of the flanges 118 shown in FIG. 13, each such hole 132 having an electrical contact 130 installed therein. Separate (e.g., electrically isolated) electrically conductive contact surfaces must be provided on the top surface of the dressing in such an embodiment (e.g., one under each sensor housing 200 or flange 118, as the case may be) to properly provision the electrical circuit shown in FIG. 15. Port 0 Pin 5 and Port 0 Pin 6 are configured as External IRQs 10 and 11 to enable open/short circuit conditions for each pair of electrical contacts 130.

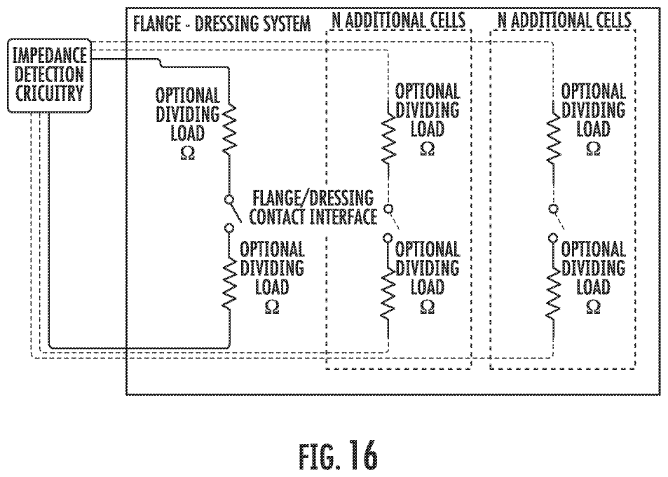

FIG. 16 schematically shows another electrical circuit for an example embodiment of a decannulation detection system for monitoring electrical continuity between a tracheostomy tube assembly (e.g., 100, 101, 102, see FIGS. 1 and 5-13) and an electrically conductive dressing (e.g., 10, see FIGS. 2 and 3) positioned (e.g., adhesively) about and/or around the wound or incision site of a patient. The detection of electrical continuity (e.g., by flow of electrical current through an electrical circuit) may be intermittently monitored or continuously monitored. The electrical coupling between the tracheostomy tube assembly 100, 101, 102 and the dressing 10 may be in the form of resistive and/or reactant impedance to infer a position of such tracheostomy tube assembly 100, 101, 102 relative to the dressing 10. When the conductive element(s) 130 in the tracheostomy tube assembly 100, 101, 102 separate from (e.g., become spaced apart from, and no longer in direct contact with, such as occurs in the case of an open circuit or electrical discontinuity) the conductive elements (e.g., wires 140-1, 140-2, 140-3, see FIGS. 2 and 3) in the dressing 10, an infinite impedance condition occurs and the total impedance of the electrical circuit is altered. As shown in FIG. 16, such an electrical circuit may comprise several discrete “cells” (e.g., electrical sub-circuits that are connected in parallel to each other) to provide additional information on the interaction and/or relative movement between the tracheostomy tube assembly 100, 101, 102 and the dressing 10. One or more infinite impedance conditions would modify the overall impedance of the electrical circuit, as detected by the system, and, as such, would trigger a warning regarding the potential occurrence of a partial or full decannulation event. In some embodiments, the electrical circuit shown in FIG. 16 includes voltage divider components to regulate power consumption.

Further configurations of suitable electric circuits in addition to those shown in FIGS. 14-16 will be readily understood by persons having ordinary skill in the art.

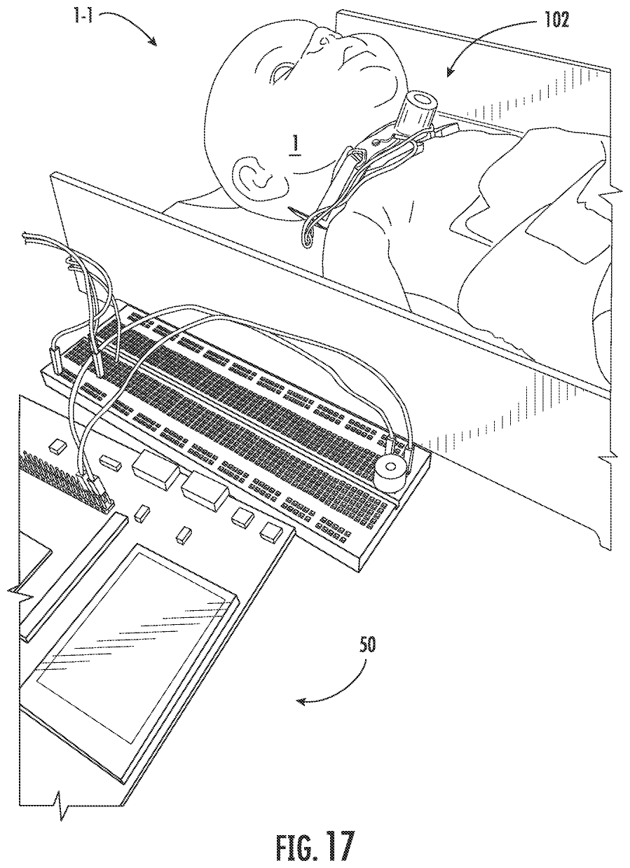

FIG. 17 shows the tracheostomy tube assembly 102 of FIG. 13 attached about the neck of a simulated pediatric patient 1 as part of a decannulation detection system, generally designated 1-1. The decannulation detection system 1-1 comprises a controller, generally designated 50, to monitor the impedance of the electrical circuit formed in the decannulation detection system 1-1 and, when the impedance of the electrical circuit changes, to trigger an alert regarding the possible occurrence of a decannulation event.

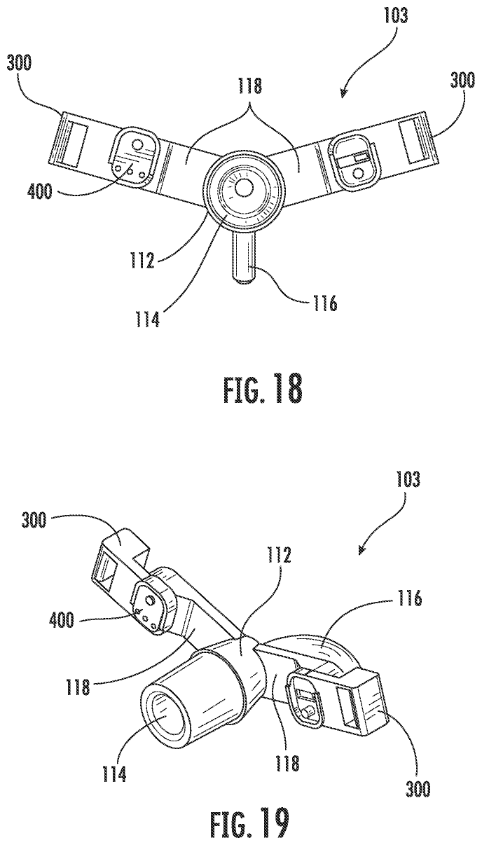

FIGS. 18 and 19 show various aspects of another example embodiment of a tracheostomy tube assembly, generally designated 103, for use in a decannulation detection system by detecting light at a photosensor 400 attached to one of the flanges 118 of the tracheostomy tube assembly 103. The features of the tracheostomy tube assembly 103 are generally similar to the features of the tracheostomy tube assembly 101 and features that are not specifically described herein as being different with respect to the tracheostomy tube assembly 103 are substantially identical to the same features of the tracheostomy tube assembly 101.

A decannulation detection system that is based on light detection can use an active light source, such as an LED, and/or can rely on ambient light.

In either embodiment, a light sensor 400 is provided to monitor for the presence or absence of light that is incident upon the light sensor 400. When the amount (e.g., intensity) of light incident on the light sensor 400 exceeds a predetermined threshold that has been determined to be correlated to the possible occurrence of a partial or complete decannulation event, the decannulation detection system is configured to trigger an alert to notify caregivers of the possible occurrence of a decannulation event. As shown, the tracheostomy tube assembly 103 may be configured to have mounted thereto a plurality of light sensors 400. These light sensors 400 can be configured to operate substantially identically to each other or different from each other, such that one or both light sensors 400 could be active, one or both light sensors 400 could be passive, or one light sensor 400 could be active and the other light sensor 400 could be passive.

In embodiments in which one or more light sensors 400 are configured as an active light-detection sensor, the decannulation detection system can use one or more active light sources, or light emitters. The light emitters can be configured to emit light having a designated wavelength of light that corresponds to a wavelength of light that the photosensor 442 is configured to detect. This designated wavelength of light can be a wavelength outside of the visible light spectrum and, in some instances, can be a wavelength of light that is not found in ambient light on Earth. One or more such light emitters can be paired with a corresponding one or more of the light sensors 400, so as to advantageously improve the performance of such light-detection sensors 400 having an active configuration. Thus, each light sensor 400 can have associated therewith one or more light emitters. The use of one or more active light source for each light sensor 400 provides a more robust light-detection method, as filters and/or wavelength selection can be used (e.g., by a controller and/or by the light sensor 400 itself) to reduce the probability of false positives or false negatives. The use of an active light source provides the additional safeguard for operation of the decannulation detection system in low-light environments (e.g. nighttime, dark room, or for instances where clothing may be worn by the patient that might block ambient light from reaching the photosensor even during a decannulation event).

As shown in FIGS. 18 and 19, the tracheostomy tube assembly 103 comprises light sensor housings 300 that are attached (e.g., rigidly, removably, permanently, or in any suitable manner) to a conventional tracheostomy tube holder, much in the same manner by which the contact switch sensor housings 200 were described as being attached to a conventional tracheostomy tube holder in the tracheostomy tube assembly 101 shown in FIGS. 5-12, thereby allowing for a retrofit application of known tracheostomy tube holders as part of such a decannulation detection system. The tracheostomy tube assembly 103 shown in FIGS. 18 and 19 is a BIVONA® Pediatric Tracheostomy Tube.

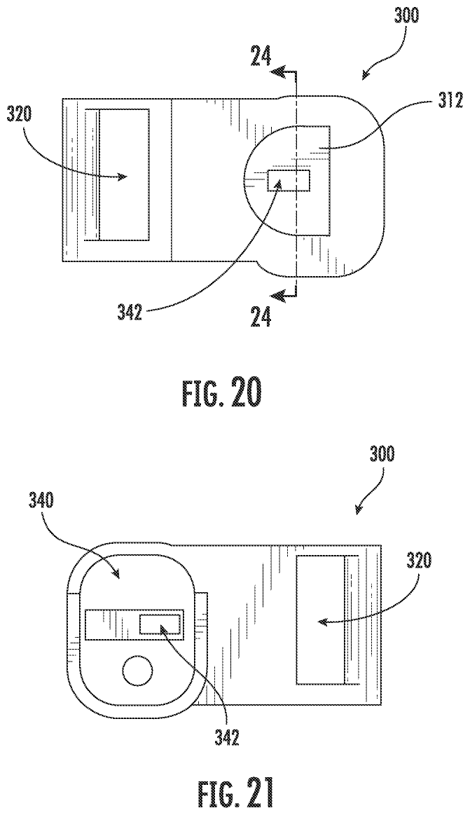

In order for such a known tracheostomy tube holder to be used as part of a decannulation detection system, a light sensor housing 300 is attached (e.g., in a press-fit, or interference fit, manner, or via any other suitably rigid attachment type) by insertion through one of the tie holes formed through the flange 118, thereby forming a quasi-extension of the flange 118. Aspects of the light sensor housing 300 are further illustrated in FIGS. 20-25, which will be described in further detail hereinbelow.

FIGS. 20 and 21 are respective front and rear views of an example embodiment of a light sensor housing 300. On one end of the light sensor housing 300, a protrusion 312 is formed. The protrusion 312 has a substantially similar cross-sectional shape to that of the tie holder formed in the conventional tracheostomy tube holder, such that the protrusion 312 can be inserted within the tie holder of the conventional tracheostomy tube holder and secured therein (e.g., via an interference fit or other suitably rigid mechanical attachment type). A cavity with a slot that extends entirely through the entire thickness of the protrusion. This cavity, generally designated 340, and a slot, generally designated 342, are formed through this protrusion 312 and are shaped to receive a light sensor 400 (e.g., any suitable photosensor) therein, with the photoreceptor 442 thereof being positioned within the slot 342. In the example embodiment shown, the light sensor housing 300 provides a slot 342 with an extruded column to mate with the central through-hole test point (e.g., photoreceptor 442) of the ADAFRUIT® ALS-PT19 Analog Light Sensor Breakout Board (e.g., the light sensor 400). Any suitable photosensitive device or element may be used for the light sensor 400. On an end of the light sensor housing 300, opposite from the end where the protrusion 312 is formed, the light sensor housing 300 comprises a slot, generally designated 320, for attachment of a tracheostomy tube tie to secure the tracheostomy tube assembly 300 in a substantially fixed position over the stoma.

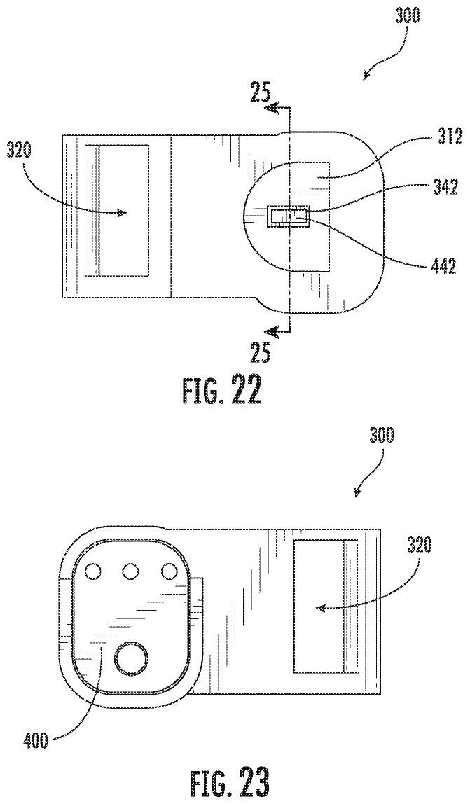

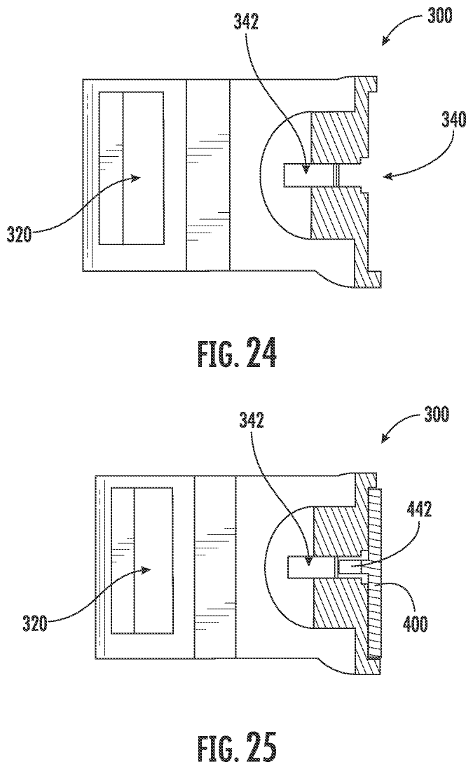

As shown in FIGS. 22 and 23, a light sensor 400 (e.g., ADAFRUIT® ALS-PT19 Analog Light Sensor Breakout Board) is inserted within the cavity 340 and aligned such that the photosensor 442 of the light sensor 400 is formed in the light sensor housing 300. In the example embodiment shown, one light sensor 400 can be installed per light sensor housing 300. FIG. 24 is a cross-sectional view taken along cut-plane 24-24 in FIG. 20, in which the light sensor 400 is omitted to show other features of the light sensor housing 300. FIG. 25 is a cross-sectional view taken along cut-plane 25-25 in FIG. 22 and shows various aspects of the light sensor 400 shown in FIGS. 22 and 23. As shown in FIG. 25, the light sensor 400 is installed within the cavity 340 and slot 342 formed through the protrusion 312 of the light sensor housing 300. As shown in FIG. 25, the photosensor 442 does not protrude entirely through the slot 342, but is instead recessed within the slot 342. In some embodiments, the outer surface of the photosensor 442 may be coplanar with the outermost edge of the slot 342 (e.g., coplanar with where the slot 342 terminates at the surface of the protrusion 312 inserted within the tie slot of the tracheostomy tube holder. The slot 342, which can also be referred to as an aperture, is positioned to allow light to reach (e.g., be incident upon) the photosensor 442 of the light sensor 400.

In some embodiments, the flanges 118 and/or the light sensor housings 300 comprise a deformable material (e.g., silicone or similar suitable material) that is configured to conform to the surface of the dressing 10 or to the skin of the patient (e.g., in instances in which the tracheostomy tube holder 103 may be used in a configuration in which the light sensors 400 are positioned to extend beyond the perimeter of the dressing 10, this deformable aspect allowing for ambient light to be blocked from being incident upon the photosensor 442 of the light sensor 400 when the tracheostomy tube assembly 103 is pressed against the dressing or the skin.

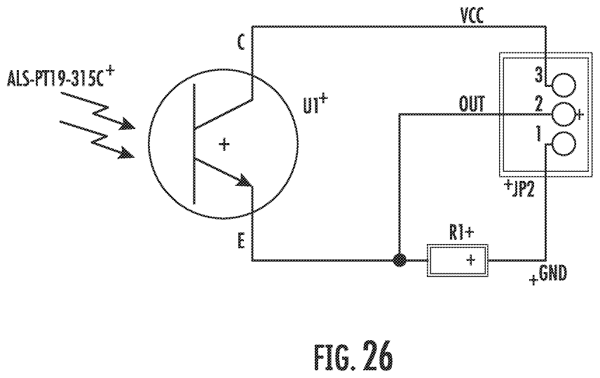

FIG. 26 shows an example embodiment of an electrical circuit that is formed for operation of the light sensor 400 of the tracheostomy tube assembly 103 shown in FIGS. 18-25. As shown in FIG. 26, The light sensors 400 do not require any additional signal conditioning or circuitry to integrate with an instrumented tracheostomy monitoring system. VCC and GND can be provided (e.g., from a Renesas S7G2 board). P000 and P001 were configured as AN00 and AN02 to provide MCU interface to the light sensor 400. This configuration allows independent detection of light by the light sensor 400 attached to both of the light sensor housings 300, so as to detect movement of the tracheostomy tube assembly 103 on both lateral sides of the central portion 112. Further configurations of suitable electrical circuits than the electrical circuit shown in FIG. 26 will be readily understood by persons having ordinary skill in the art.

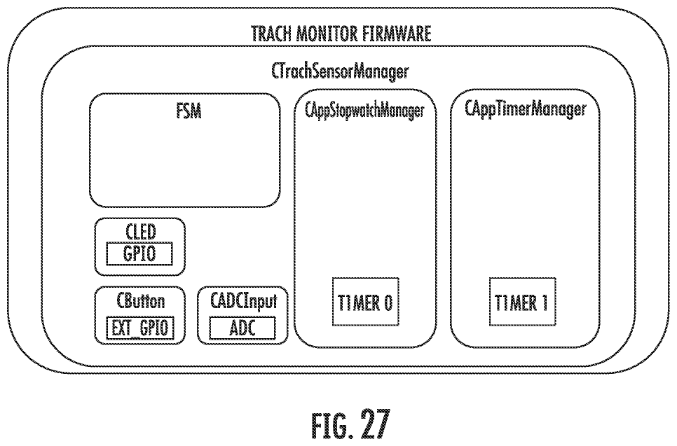

FIG. 27 is a block diagram of an example embodiment of software components in a decannulation detection system. An embedded application framework for an example embodiment of a decannulation detection system was developed to drive both contact switch and ambient light sensor methods of detection of a movement of the tracheostomy tube assembly relative to the dressing beyond a predetermined amount, or degree or distance, of movement, which can correspond to and/or be indicative of the possible occurrence of a decannulation event. The software disclosed herein is architected and configured to provide sensor agnostic high-level controls with abstraction layers that provide extensible/testable interfaces to reduce refactoring burden if and when entering IEC 13485/IEC 62304 Design Controls. Firmware was developed on a Renesas Synergy S7 SK-S7G2 Starter Kit prototyping board. In the example embodiment shown in FIG. 27, the software system comprises or consists of a single software item with underlying software units implemented as C++ classes. Hardware Abstraction Layer Drivers were provided by Renesas Software Synergy Package v1.51.

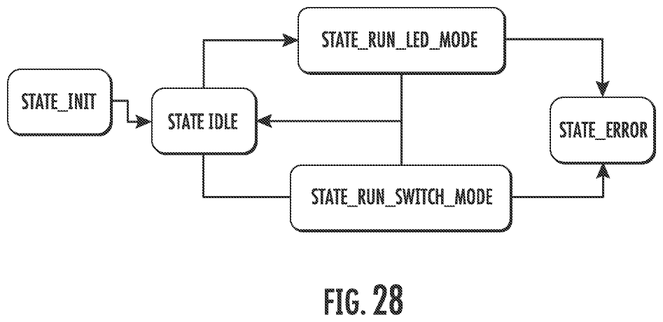

FIG. 28 is a flow chart for operation of a decannulation detection system. The Finite State Machine class implemented the event-based state machine shown in FIG. 28. In the example prototype developed, transitions between run states were triggered using the user press button (L5) on the prototype board.

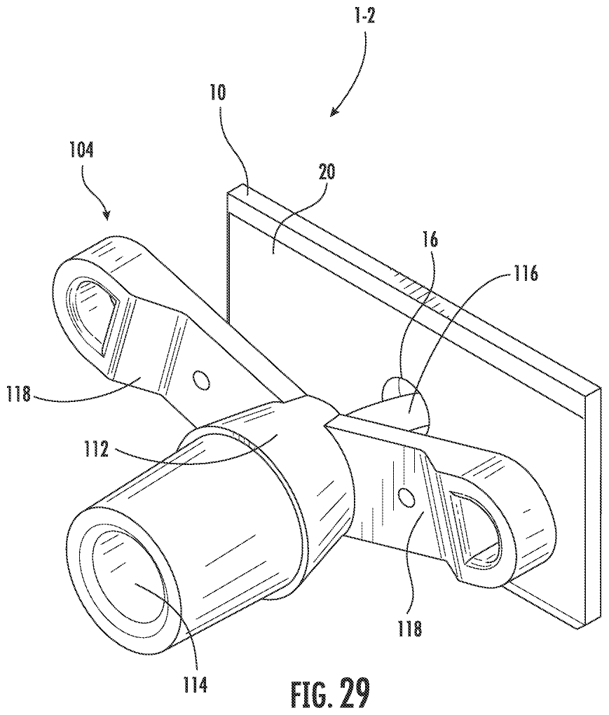

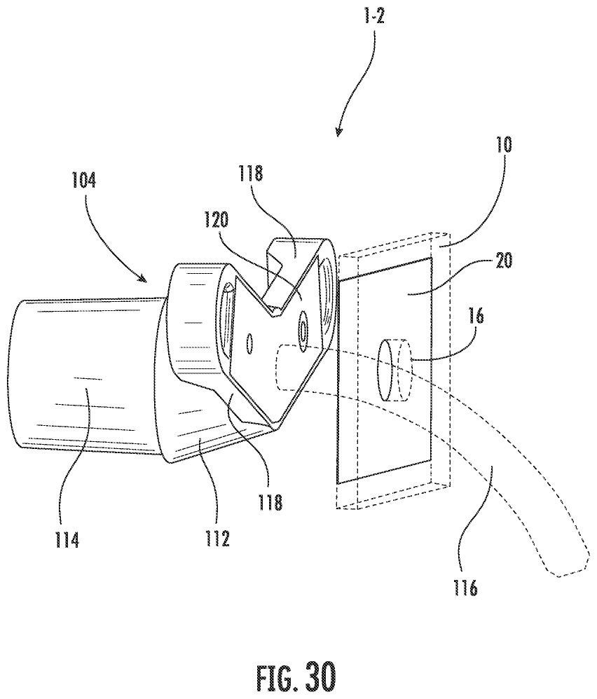

FIGS. 29 and 30 are respective perspective views of another example embodiment of a decannulation detection system, generally designated 1-2. The decannulation detection system 1-2 comprises a tracheostomy tube assembly, generally designated 104, that is configured to detect a decannulation event (e.g., a partial decannulation and/or a full decannulation) by measuring and monitoring capacitance values between two capacitive plates. A first capacitive plate 20 is attached to a dressing 10 and a second capacitive plate 120 is attached to the tracheostomy tube assembly 104. The structures of the tracheostomy tube assembly 104 shown in FIGS. 29 and 30 are substantially similar to that which is shown and described with respect to the tracheostomy tube assembly 102 shown in FIG. 13. Unless described to the contrary herein, all like structures of the tracheostomy tube assembly 104 are substantially similar, or identical, to the corresponding structures of the tracheostomy tube assembly 102. For example, the tracheostomy tube assembly 104 comprises flanges 118 that are attached, respectively, on opposite edges of a central portion 112 and extend away from each other (e.g., radially, as described elsewhere herein). The central portion 112 has attached thereto a raised portion 114 that is configured to prevent blockages of the tracheostomy tube 116 that may otherwise lead to a hypoxic condition of the patient.

The dressing 10 has a first capacitive plate 20 on an outer surface thereof (e.g., the surface that is opposite the surface by which the dressing 10 is attached, for example, adhesively, to the skin of the patient about the incision or wound site). The second capacitive plate 120 is attached to the rear surface of the central portion 112 and flanges 118, the rear surface being the surface that is opposite to the surface to which the raised portion 114 is attached to the central portion 112 and also the surface that will be adjacent to (e.g., closest to) the dressing 10 when the tracheostomy tube 116 is inserted through the incision or wound site (e.g., stoma). Thus, when the tracheostomy tube 116 is inserted within the stoma, the first and second capacitive plates 20, 120 are positioned adjacent to each other (e.g., positioned within a distance from each other to measure a change in capacitance therebetween due to relative movement between the first and second capacitive plates 20, 120, for example, towards/away from each other).

According to this example embodiment, the relative position between the first capacitive plate 20 attached to the dressing 10 and the second capacitive plate 120 attached to the tracheostomy tube assembly 104 can be determined by measuring one or more capacitance values between the first and second capacitive plates 20, 120 to detect the occurrence of a decannulation event (e.g., in realtime). In some embodiments, the first and second capacitive plates 20, 120 can each be subdivided into a plurality of capacitive plates, such that a plurality of first capacitive plates 20 are provided on the dressing 10 and a plurality of second capacitive plates 120 are provided on the tracheostomy tube assembly 104. Each of the first capacitive plates 20 is positioned on the dressing 10 such that, when the tracheostomy tube assembly 104 is positioned over the dressing 10, each such first capacitive plate 20 will be positioned adjacent to a corresponding one of the second capacitive plates, such that each of the first capacitive plates 20 forms a capacitive plate pair with one of the second capacitive plates 120, thereby allowing for capacitance to be measured between such capacitive plate pairs to advantageously detect a partial or full decannulation event that may not be recognized when using a single first capacitive plate 20 and a single second capacitive plate 120. The capacitive plate pairs may be arranged in any pattern and/or position without limitation.

In the example embodiments shown in FIGS. 29 and 30, the first capacitive plate 20 comprises a hole 16 through which the tracheostomy tube 116 is insertable. Similarly, the second capacitive plate 120 also comprises a hole through which the tracheostomy tube 116 is inserted. The tracheostomy tube 116 is shown in broken line in FIG. 30 and not passing through the hole 16 because the tracheostomy tube assembly 104 is spaced apart from the dressing 10 in this view. In embodiments in which there are a plurality of first and second capacitive plates 20, 120 (e.g., arranged as respective capacitive plate pairs), such first and second capacitive plates 20, 120 may be arranged on the tracheostomy tube assembly 104 and the dressing 10 outside of a region through which the tracheostomy tube 116 may pass, thereby obviating the need for any such holes 16 to be formed in any of the first and second capacitive plates 20, 120.

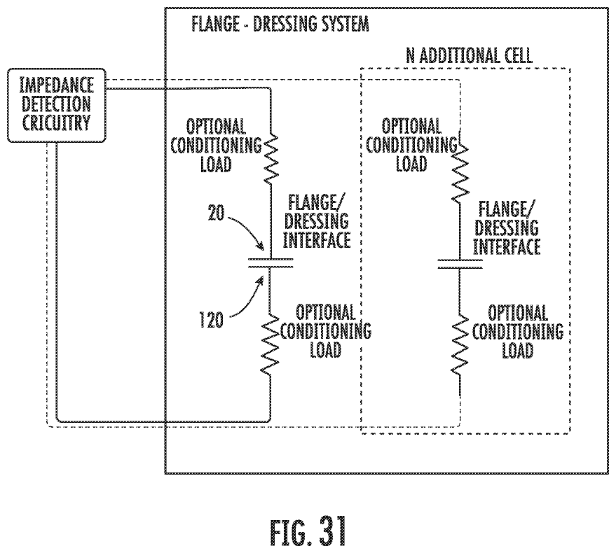

FIG. 31 shows an example schematic electrical circuit for the example embodiment of the decannulation detection system 1-2 shown in FIGS. 29 and 30. The electrical circuit shown in FIG. 31 is configured for monitoring capacitance between the first and second capacitive plates 20, 120 that are attached, respectively, to a dressing 10 and a tracheostomy tube assembly 104, the dressing 10 being positioned (e.g., adhesively) about and/or around a wound or incision site. As shown in FIG. 31, the electrical circuit monitors and measures capacitance between the first and second capacitive plates 20, 120 and uses such measured capacitance values to infer a position of the tracheostomy tube assembly 104 relative to the dressing 10. To do this, the decannulation detection system 1-2 comprises N sets of pairs of opposing first and second capacitive plates 20, 120 (e.g., a first capacitive plate 20 in or on the dressing 10 and a second capacitive plate 120 on the tracheostomy tube assembly 104, with an insulator being provided between the first and second capacitive plates 20, 120 to prevent an electrical short condition between the first and second capacitive plates 20, 120), where Nis greater than or equal to 1. Thus, the “N Additional Cell” shown in FIG. 31 is shown to illustrate an electrical circuit when there are two first capacitive plates 20 and two second capacitive plates 120. The “N Additional Circuit” can be duplicated as many times as is necessary to equal the quantity of capacitive plate pairs formed by the quantity of first and second capacitive plates 20, 120.

As the position of the second capacitive plate 120 on the tracheostomy tube assembly 104 changes relative to the first capacitive plate 20 on the dressing 10, accumulated charge varies, or is modified, this change in cumulative charge being used to infer a position of the tracheostomy tube assembly 104 relative to the dressing 10. The detection of capacitance may be intermittently monitored or continuously monitored. As shown in FIG. 31, such an electrical circuit may comprise N discrete “cells” (e.g., a plurality of electrical sub-circuits that are connected in parallel to each other, with the quantity of such electrical sub-circuits being equal to the quantity of capacitive plate pairs) to provide additional information on the interaction and/or relative movement between the tracheostomy tube assembly 104 and the dressing 10. Changes in capacitance across these N cells are monitored and used to trigger a warning regarding the potential occurrence of a partial or full decannulation event. In some embodiments, the electrical circuit shown in FIG. 31 can be modified to include voltage divider components to condition electrical current, as would be understood by persons having ordinary skill in the art.

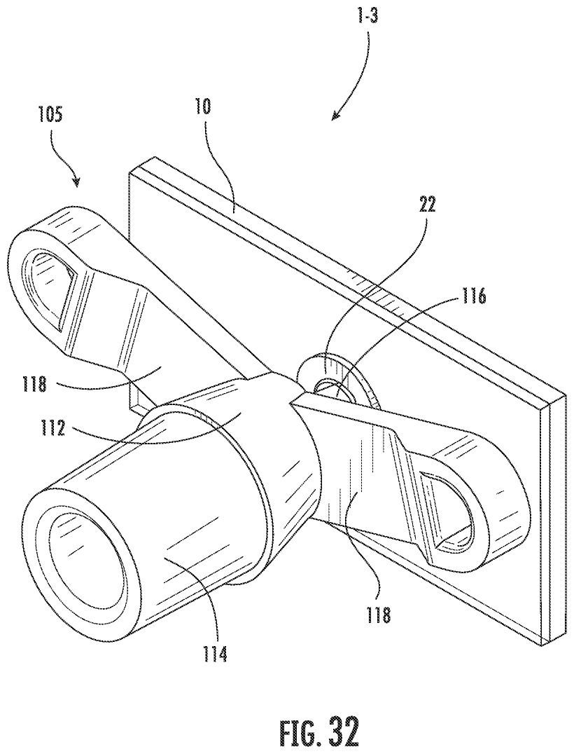

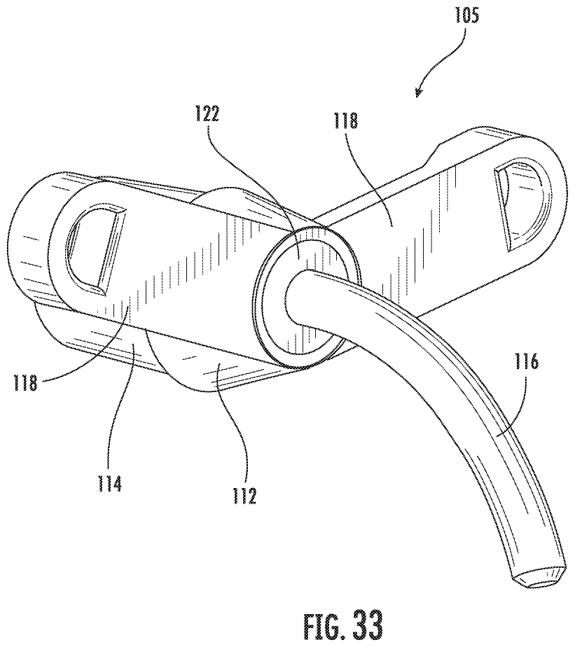

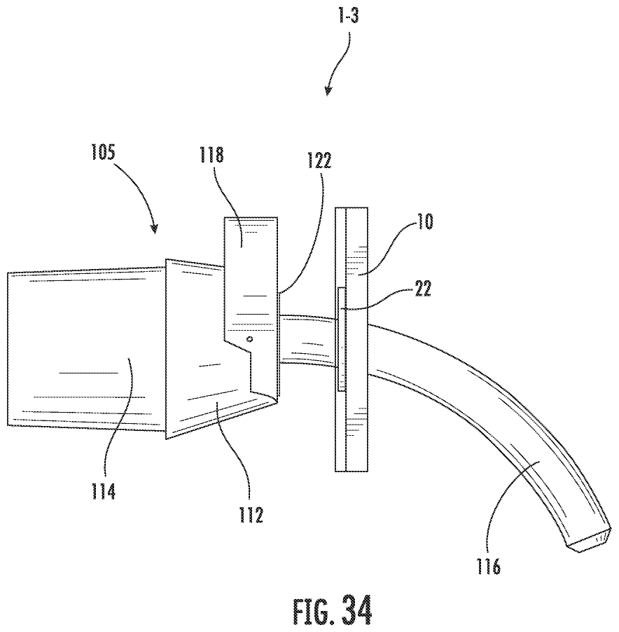

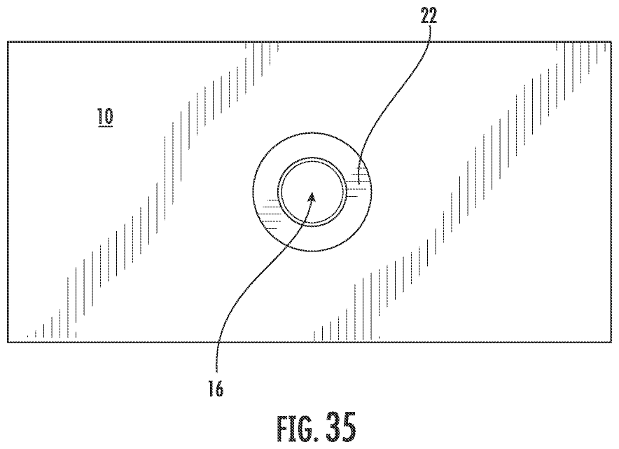

FIGS. 32-35 show various aspects of another example embodiment of a decannulation detection system, generally designated 1-3. This decannulation detection system 1-3 comprises a tracheostomy tube assembly, generally designated 105, and a dressing 10 and is configured to detect a decannulation event (e.g., a partial decannulation and/or a full decannulation) by measuring and/or monitoring inductance values between two inductively coupled structures, namely, a first inductor 22 and a second inductor 122. The first and second inductors 22, 122 can be in the form of, for example, coils (e.g., flattened coils of wire) of one or more electromagnets, permanent magnets, inductance sensors, and the like, including combinations thereof, without limitation. The structures of the tracheostomy tube assembly 105 shown in FIGS. 32-34 are substantially similar to that which is shown and described with respect to the tracheostomy tube assemblies 102, 104 shown in FIGS. 13 and 29-30, respectively. Unless described to the contrary herein, all like structures of the tracheostomy tube assembly 105 are substantially similar, or identical, to the corresponding structures of the tracheostomy tube assemblies 102, 104. For example, the tracheostomy tube assembly 105 comprises flanges 118 that are attached, respectively, to opposite edges of a central portion 112 and extend radially away from each other. The central portion 112 has attached thereto a raised portion 114 that is configured to prevent blockages of the tracheostomy tube 116 that may otherwise lead to a hypoxic condition of the patient.

The dressing 10 has a first inductor 22 on an outer surface thereof (e.g., the surface that is opposite the surface by which the dressing 10 is attached, for example, adhesively, to the skin of the patient about the incision or wound site). This first inductor 22 can be, for example, in the form of an inductance source, such as a plurality of flattened coils in the form of an electromagnet. The first inductor 22 is shown being concentrically arranged about the hole 16 in the dressing 10 through which the tracheostomy tube 116 is inserted for insertion through the wound and/or incision site (e.g., into the stoma). However, the first inductor 22 is not limited to the position, size, and/or shape shown in FIGS. 32-35 and can be implemented having any suitable shape, size, and/or position. A second inductor 122 (e.g., an inductance sensor) is attached to the rear surface of the central portion and flanges, the rear surface being the surface that is opposite to the surface to which the raised portion 114 is attached to the central portion 112 and also the surface that will be adjacent to (e.g., closest to) the dressing 10 when the tracheostomy tube 116 is inserted through the incision or wound site (e.g., stoma). The second inductor 122 is shown as concentrically arranged about the tracheostomy tube 116 but is not limited to this position and/or shape. However, the second inductor 122 is not limited to the position, size, and/or shape shown in FIGS. 32-35 and can be implemented having any suitable shape, size, and/or position. Thus, when the tracheostomy tube 116 is inserted into or through the stoma, the first and second inductors 22, 122 are adjacent to each other (e.g., positioned within a distance from each other that allows for measurement and/or detection of a change in inductance, or magnetic field, between the first and second inductors 22, 122 due to relative movement between the first and second inductors 22, 122.

In the example embodiment shown, the first and second inductors 22, 122 are flattened coils formed out of wire. The first or second inductors 22, 122 can be continuously or intermittently provided with an electric current through an electrical circuit connected thereto. The electric current provided to the first and/or second inductors 22, 122 can be variable or constant without limitation. The first and second inductors 22, 122 are substantially coaxially aligned with each other when the tracheostomy tube assembly 105 is positioned against, or adjacent to, the dressing 10, with the tracheostomy tube 116 being inserted within and through the stoma. The electric current flowing through the first and/or second inductors 22, 122 (e.g., whichever is the “active,” or transmitting, inductor) will cause the corresponding first and/or second inductor 22, 112 that receives the electric current to generate a magnetic field that is oriented so as to induce a corresponding voltage and electrical current in the other of the first and/or second inductors 22, 122 (e.g., the “passive,” or receiving, inductor).

Thus, in some instances, the decannulation detection system 1-3 can be configured such that the first inductor 22 is provided with an electrical current and, thus, also considered as the “active” inductor, in which case the first inductor 22 generates a magnetic field that is oriented so as to induce a corresponding voltage and electrical current in the second inductor 122, which is thus considered as the “passive” inductor, in the manner of an inductance sensor. Furthermore, in some other instances, the decannulation detection system 1-3 can be configured such that the second inductor 122 is provided with an electrical current and, thus, also considered as the “active” inductor, in which case the second inductor 122 generates a magnetic field that is oriented so as to induce a corresponding voltage and electrical current in the first inductor 22, which is thus considered as the “passive” inductor, in the manner of an inductance sensor.

Regardless of the configuration and which of the first and second inductors 22, 122 is regarded as the “active” inductor and which is regarded as the “passive” inductor, the current and voltage induced in the “passive” inductor is measured and/or monitored and used to trigger an alert when a change in the voltage and/or current induced in the “passive” inductor is determined to be outside of a predetermined range, or greater than or less than a threshold value associated with the voltage and/or the current. This predetermined range or threshold value for the voltage and/or current is correlated with (e.g., in a calibrated, precise manner) a movement of the tracheostomy tube assembly 104 away from the dressing 10 that has been determined empirically (e.g., during design of the decannulation detection 1-3 for a particular size, configuration, etc.) to be indicative of the possible occurrence and/or initiation of a decannulation event. In some embodiments, a transfer function (e.g., impedance) can be used as a proxy to infer a distance between the tracheostomy tube assembly 105 and the dressing 10.

According to the example embodiment of the decannulation detection system 1-3, the relative position between the first inductor 22 attached to the dressing 10 and the second inductor 122 attached to the tracheostomy tube assembly 105 can be determined by measuring one or more inductance parameters (e.g., of a magnetic field produced by the inductor) to detect the occurrence of a decannulation event (e.g., in realtime).

In some embodiments, a plurality of first inductors 22 can be provided on the dressing 10 and a plurality of second inductors 122 can be provided on the tracheostomy tube assembly 105 and arranged, with respect to each other, to form inductor pairs, in which the first and second inductors 22, 122 of each such inductor pair are substantially coaxial with each other when the tracheostomy tube assembly 105 is in an installed position, against or adjacent to the dressing 10. These inductor pairs can thus be provided at a plurality of different positions on the tracheostomy tube assembly 105 and dressing 10, respectively, such that inductance between the first and second inductors 22, 122 can be measured between each of the inductor pairs to advantageously more readily detect a partial or full decannulation event than may be possible using a single first inductor 22 and a single second inductor 22. While one or more of the first and second inductors 22, 122 of the respective pluralities thereof can be provided around (e.g., coaxial with) the hole 16, in embodiments of the decannulation detection system 1-3 in which such inductor pairs are provided, it is possible in some configurations for none of the inductor pairs to be positioned around the hole 16, whether coaxially arranged or otherwise; instead, in some instances, all of the inductor pairs may be spaced apart from the hole 16, such that none of the inductor pairs is arranged coincident or around any of the hole 16, of any portion thereof. The inductor pairs may be arranged in any pattern and/or position without limitation. In some embodiments having a plurality of first inductors 22 and a plurality of second inductors 122, in some of the inductor pairs the first inductor 22 thereof may be the “active” inductor and the second inductor 122 thereof may be the “passive” or sensing inductor and, in the other inductor pairs, the first inductor 22 thereof may be the “passive” or sensing inductor and the second inductor 122 thereof may be the “active” inductor.

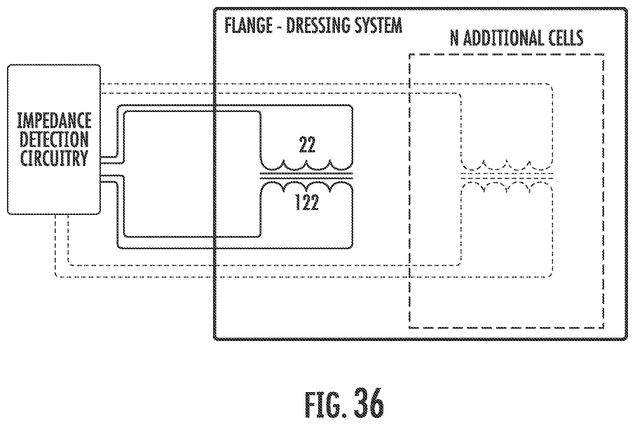

FIG. 36 shows an example embodiment of electrical circuit for the example embodiment of the decannulation detection system 1-3 shown in FIGS. 32-35. The electrical circuit shown in FIG. 36 is configured for monitoring inductance between the first and second inductors 22, 122 that are attached, respectively, to a dressing 10 and a tracheostomy tube assembly 104, the dressing 10 being positioned (e.g., adhesively) about and/or around a wound or incision site. As shown in FIG. 36, the electrical circuit monitors and measures inductance (e.g., one or more parameters associated with a magnetic field) between the first and second inductors 22, 122, this inductance being generated by an inductance source (i.e., one of the first and second inductors 22, 122) as shown in the example embodiment of the decannulation detection system 1-3 of FIGS. 32-35. In some embodiments, the first inductor 22 or the second inductor 122, whichever is to be regarded as the “active” inductor in an inductor pair, may be replaced with a permanent magnet, so that the decannulation detection system 1-3 may remain operational without the requirement of provision of electrical power to the “active” inductor for generating the magnetic field that induces the voltage and electric current in the “passive” or sensing inductor. The inductance parameter(s) measured by the second inductance element is/are correlated to relative positions of the tracheostomy tube assembly 105 (e.g., especially at the second inductor 122) and the dressing 10 (e.g., especially at the first inductor 22) and the inductance parameter(s) are used to infer a position of the tracheostomy tube assembly 105 relative to the dressing 10 and/or a relative movement of the tracheostomy tube assembly 105 away from the dressing 10.

In some embodiments, the decannulation detection system 1-3 can comprise N (e.g., a plurality) of each of the first and second inductors 22, 122, in the manner of inductor pairs described elsewhere herein. These inductor pairs can be, for example, in the form of respective pairs of concentric coils of electrically conductive material. As the position of the second inductor 122 on the tracheostomy tube assembly 105 changes relative to the first inductor 22 on the dressing 10, a change in inductance (e.g., mutual inductance) is detected. This change in inductance is then used to infer a position of the tracheostomy tube assembly 105 relative to the dressing 10. The detection of inductance may be intermittently monitored or continuously monitored. As shown in FIG. 36, such an electrical circuit may comprise N discrete “cells” (e.g., a plurality of electrical sub-circuits that are connected in parallel to each other, each electrical sub-circuit comprising an inductor pair having a single first inductor 22 and a single second inductor 122) to provide additional information on the interaction and/or relative movement between the tracheostomy tube assembly 105 and the dressing 10. Changes in mutual inductance across the first and second inductors 22, 122 in each of these N cells are monitored and used to trigger a warning regarding the potential occurrence of a partial or full decannulation event. In some embodiments, the electrical circuit shown in FIG. 36 includes voltage divider components to condition electrical current. The positions of the first and second inductors 22, 122 can be reversed from that which is shown in the decannulation detection system 1-3 FIGS. 32-35 and either of the first or second inductors 22, 122 can be used as the inductance source, or emitter, without limitation.

Each of the example embodiments of the decannulation detection systems and/or tracheostomy tube assemblies disclosed herein is suitable for use in detecting dislodgement of a tracheostomy tube from a wound or incision cite before a full decannulation event has occurred.

Each of the example embodiments of the decannulation detection systems and/or tracheostomy tube assemblies disclosed herein is able to adapt to various types of conventional pediatric tracheostomy tubes.

Each of the example embodiments of the decannulation detection systems and/or tracheostomy tube assemblies disclosed herein is low-profile, meaning that each of the respective tracheostomy tube assemblies is not prevented from sitting flush against the patient's neck dressing 10 and/or skin.

Each of the example embodiments of the decannulation detection systems and/or tracheostomy tube assemblies disclosed herein is suitable for use in a home healthcare setting.

Each of the example embodiments of the decannulation detection systems and/or tracheostomy tube assemblies disclosed herein have been evaluated to demonstrate the ability to detect partial and/or complete decannulation of a tracheostomy tube. Ambient light detection (e.g., in the manner of tracheostomy tube holder 103) has been determined to have high sensitivity, along with a correspondingly higher potential for false positives, while the use of a spring pin (e.g., in the manner of tracheostomy tube assemblies 101, 102) and detection of electrical continuity has slightly less sensitivity and is a binary signal.

In each of the example embodiments of the decannulation detection systems and/or tracheostomy tube assemblies disclosed herein, the sensor housings (e.g., 200, 300) sit flush with the surface of the flange 118 of the tracheostomy tube assembly (e.g., 101, 103).

Each of the example embodiments of the decannulation detection systems and/or tracheostomy tube assemblies disclosed herein is configured to detect when the flange 118 of the tracheostomy tube assembly 100-105 is no longer flush with the dressing 10. In some embodiments, the decannulation detection systems disclosed herein are configured to distinguish between displacement of a sensor on any or all of the flanges of the tracheostomy tube assembly.

In each of the example embodiments of the decannulation detection systems and/or tracheostomy tube assemblies disclosed herein, the tracheostomy tube assemblies are able to be fastened with conventional tracheostomy collar fastening methods (e.g., hook-and-loop collar, etc.).

While the decannulation detection systems described in the example embodiments disclosed herein pertain to decannulation of a tracheostomy tube, such decannulation detection systems are not limited to such applications. To the contrary, such decannulation detection systems can be applied to detect decannulation of any tube, wire, etc. inserted through an orifice or incision formed in the body of a patient (e.g., a human, but also including other animals) for which unintended removal thereof would advantageously be guarded against or prevented and/or would benefit from early warning of possible partial and/or complete decannulation thereof.

While the term “tracheostomy tube assembly” is used to illustrate an example application of the decannulation detection systems disclosed herein, such decannulation detection systems are not limited to tracheostomy tube applications. In fact, the decannulation detection systems disclosed herein can be used to detect decannulation of essentially any—ostomy tube from a wound or incision site without limitation.

The description herein describes embodiments of the presently disclosed subject matter, and in some cases notes variations and permutations of such embodiments. This description is merely exemplary of the numerous and varied embodiments. The description or mentioning of one or more representative features of a given embodiment is likewise exemplary. Such an embodiment can typically exist with or without the feature(s) mentioned; likewise, all of the features disclosed in each of the example embodiments can be applied to any of the other example embodiments disclosed herein without limitation and without regard to whether the combination of such features is expressly described herein, unless a preclusion against the combination of such features is expressly noted herein.

It will be understood that various details of the presently disclosed subject matter may be changed without departing from the scope of the presently disclosed subject matter. Furthermore, the foregoing description of the example embodiments disclosed herein is only provided for the purpose of illustration only and is not to be used to limit in any way the scope of the subject matter disclosed herein.

1. A decannulation detection system comprising:
a dressing for application over a wound or incision site, the dressing comprising a first electric circuit portion;
a tube assembly for positioning in contact with the dressing, the tube assembly comprising a second electric circuit portion; and
a controller;
wherein, when the tube assembly is in contact with the dressing, the first and second electrical circuit portions form a complete electric circuit;
wherein the controller is electrically connected to the complete electric circuit and is configured to monitor electrical continuity in the complete electric circuit; and
wherein, when the controller detects an electrical discontinuity in the complete electric circuit, the controller is configured to trigger an alert regarding a possible occurrence of a decannulation event.

2. The decannulation detection system of claim 1, wherein at least a portion of the tube assembly extends at least partially within a wound or incision at the wound or incision site.

3. The decannulation detection system of claim 2, wherein the wound or incision comprises a stoma and the tube assembly comprises a tracheostomy tube assembly.

4. The decannulation detection system of claim 1, wherein:
the first electrical circuit portion comprises a plurality of discontinuous wires, or foil sheets that are formed in or on an outer surface of the dressing, the outer surface being opposite a surface of the dressing that is against the skin of a patient in which the wound or incision site is formed;
the tube assembly comprises a center portion and flanges, the flanges extending from opposite sides of the center portion;
the second electrical circuit portion comprises, formed in each of the flanges, at least one electrical contact that protrudes through a surface of a corresponding one of the flanges when the tube assembly contacts the dressing; and
when the tube assembly is in contact with the dressing, each of the one or more electrical contacts form an electrical connection between different ones of the plurality of discontinuous wires or foil sheets to form the complete electric circuit.

5. The decannulation detection system of claim 4, wherein the one or more electrical contacts comprise spring-loaded pins.

6. The decannulation detection system of claim 4, wherein, when the tube assembly moves away from the dressing during the decannulation event, at least one of the one or more electrical contacts are separated from the dressing to cause the electrical discontinuity in the complete electrical circuit.

7. The decannulation detection system of claim 6, wherein each flange comprises a plurality of electrical contacts spaced apart from each other in an anticipated direction of movement or rotation of the tube assembly away from the dressing during the decannulation event.

8. The decannulation detection system of claim 1, wherein the complete electric circuit is formed only when the tube assembly is in direct contact with the dressing.

9. A method for detecting a decannulation event, the method comprising:
providing a controller;
applying a dressing over a wound or incision site, the dressing comprising a first electric circuit portion;
positioning a tube assembly adjacent to the dressing, the tube assembly comprising a second electric circuit portion, wherein, when the tube assembly is in contact with the dressing, the first and second electric circuit portions form a complete electric circuit;
connecting the controller to the complete electric circuit;
monitoring, using the controller, electrical continuity in the complete electric circuit; and
triggering, via the controller, an alert regarding a possible occurrence of the decannulation event when the controller detects the electrical discontinuity in the complete electric circuit.

10. The method of claim 9, wherein at least a portion of the tube assembly extends at least partially within a wound or incision at the wound or incision site.

11. The method of claim 10, wherein the wound or incision comprises a stoma and the tube assembly comprises a tracheostomy tube assembly.

12. The method of claim 9, wherein the electrical discontinuity in the complete electric circuit occurs only when the tube assembly has moved at least a predetermined distance away from the dressing, the predetermined distance being correlated with the possible occurrence of the decannulation event.

13. The method of claim 9, wherein:
the first electrical circuit portion comprises a plurality of discontinuous wires, or foil sheets that are formed in or on an outer surface of the dressing, the outer surface being opposite a surface of the dressing that is against the skin of a patient in which the wound or incision site is formed;
the tube assembly comprises a center portion and flanges, the flanges extending from opposite sides of the center portion;
the second electrical circuit portion comprises, formed in each of the flanges, at least one electrical contact that protrudes through a surface of a corresponding one of the flanges when the tube assembly contacts the dressing; and
when the tube assembly is in contact with the dressing, each of the one or more electrical contacts form an electrical connection between different ones of the plurality of discontinuous wires or foil sheets to form the complete electric circuit.

14. The method of claim 13, wherein the one or more electrical contacts comprise spring-loaded pins.

15. The method of claim 13, wherein, when the tube assembly moves away from the dressing during the decannulation event, at least one of the one or more electrical contacts are separated from the dressing to cause the electrical discontinuity in the complete electrical circuit.

16. The method of claim 15, wherein each flange comprises a plurality of electrical contacts spaced apart from each other in an anticipated direction of movement or rotation of the tube assembly away from the dressing during the decannulation event

17. The method of claim 9, wherein the complete electric circuit is formed only when the tube assembly is in direct contact with the dressing.

18. A decannulation detection system comprising:
a tube assembly for positioning in contact with a dressing applied over a wound or incision site or over skin at the wound or incision site, wherein the tube assembly at least partially extends within a wound or incision at the wound or incision site and comprises one or more light sensors; and
a controller;
wherein the one or more light sensors are positioned on the tube assembly such that light can only be received by the one or more light sensors from a side of the tube assembly that is positioned against the dressing or skin at the wound or incision site;
wherein the tube assembly is configured such that, when pressed against the dressing or the skin, no ambient light from outside the tube assembly can be received by the one or more light sensors;
wherein, during a decannulation event, the tube assembly is shifted away from the dressing or skin, so as to allow the ambient light to be received by the one or more light sensors; and
wherein, when any of the one or more light sensors detects light above a predetermined threshold corresponding to the decannulation event, the controller is configured to trigger an alert regarding a possible occurrence of a decannulation event.

19. The decannulation detection system of claim 18, wherein the wound or incision comprises a stoma and the tube assembly comprises a tracheostomy tube assembly.

20. The decannulation detection system of claim 18, wherein:
the tube assembly comprises a center portion and flanges, the flanges extending from opposite sides of the center portion;
each flange comprises a cavity with a slot formed therein, the slot being formed to extend from the cavity through a surface of the flange that contacts the dressing or the skin;
the one or more light sensors comprises a plurality of light sensors, each of the plurality of light sensors being attached to one of the flanges;
a photosensor of each light sensor is aligned with the slot of the flange to which such light sensor is attached; and
the flanges of the tube assembly comprise a deformable material configured to conform to the surface of the dressing or the skin to block the ambient light from the photosensor of the light sensor when the tube assembly is pressed against the dressing or the skin.

21. The decannulation detection system of claim 20, wherein the deformable material comprises silicone.

22. The decannulation detection system of claim 20, wherein, when the tube assembly moves away from the dressing during the decannulation event, one or more of the flanges are separated from the dressing or the skin to allow the ambient light to be incident on the photosensor of a corresponding one of the light sensors.

23. The decannulation detection system of claim 18, comprising a light emitter configured to emit a designated wavelength of light, wherein, during the decannulation event, the light sensor is configured to receive light from the light emitter to cause the controller to issue the alert regarding the occurrence of the decannulation event.

24. The decannulation detection system of claim 23, wherein the decannulation detection system is configured to detect the decannulation event in an environment in which no ambient light is present.

25. The decannulation detection system of claim 18, wherein the tube assembly is in direct contact with the dressing or the skin.

26. A method for detecting a decannulation event, the method comprising:
providing a controller;
attaching one or more light sensors on the tube assembly;
positioning the tube assembly in contact with a dressing applied over a wound or incision site or over skin at the wound or incision site, wherein the tube assembly at least partially extends within a wound or incision at the wound or incision site and comprises one or more light sensors, wherein:
the one or more light sensors are positioned on the tube assembly such that light can only be received by the one or more light sensors from a side of the tube assembly that is positioned against the dressing or skin at the wound or incision site;
when pressed against the dressing or the skin, no ambient light from outside the tube assembly can be received by the one or more light sensors due to the tube assembly being pressed against the dressing or the skin; and
during a decannulation event, the tube assembly is shifted away from the dressing or skin, so as to allow the ambient light to be received by the one or more light sensors;
detecting, using the one or more light sensors, light; and
triggering, via the controller, an alert regarding a possible occurrence of the decannulation event when any of the one or more light sensors detects light above a predetermined threshold corresponding to the decannulation event.

27. The method of claim 26, wherein the wound or incision comprises a stoma and the tube assembly comprises a tracheostomy tube assembly.

28. The method of claim 26, wherein light is detected by the one or more light sensors only when the tube assembly has moved at least a predetermined distance away from the dressing, the predetermined distance being correlated with the possible occurrence of the decannulation event.

29. The method of claim 26, wherein:
the tube assembly comprises a center portion and flanges, the flanges extending from opposite sides of the center portion;
each flange comprises a cavity with a slot formed therein, the slot being formed to extend from the cavity through a surface of the flange that contacts the dressing or the skin;
the one or more light sensors comprises a plurality of light sensors, each of the plurality of light sensors being attached to one of the flanges;
a photosensor of each light sensor is aligned with the slot of the flange to which such light sensor is attached; and
the flanges of the tube assembly comprise a deformable material configured to conform to the surface of the dressing or the skin to block the ambient light from the photosensor of the light sensor when the tube assembly is pressed against the dressing or the skin.

30. The method of claim 29, wherein the deformable material comprises silicone.

31. The method of claim 29, wherein, when the tube assembly moves away from the dressing during the decannulation event, one or more of the flanges are separated from the dressing or the skin to allow the ambient light to be incident on the photosensor of a corresponding one of the light sensors.

32. The method of claim 26, comprising emitting, from a light emitter, light having a designated wavelength, wherein, during the decannulation event, the light sensor receives the light having the designated wavelength from the light emitter to cause the controller to issue the alert regarding the occurrence of the decannulation event.

33. The method of claim 32, wherein the decannulation detection system can detect the decannulation event in an environment in which no ambient light is present.

34. The method of claim 26, wherein the tube assembly is in direct contact with the dressing or the skin.

35. A decannulation detection system comprising:
a dressing for application over a wound or incision site, the dressing comprising one or more first capacitive plates;
a tube assembly for positioning in contact with the dressing and comprising one or more second capacitive plates;
an electrical circuit that is connected to the one or more first capacitive plates and the one or more second capacitive plates; and
a controller that is configured to detect and monitor a capacitance between the one or more first capacitive plates and the one or more second capacitive plates, wherein the capacitance varies based on a distance between the one or more first capacitive plates and the one or more second capacitive plates;
wherein, when the controller detects that the capacitance between the one or more first capacitive plates and the one or more second capacitive plates is not within a predetermined range of capacitance values or is above or below a threshold capacitance value, the controller is configured to trigger an alert regarding a possible occurrence of a decannulation event.

36. The decannulation detection system of claim 35, wherein at least a portion of the tube assembly extends at least partially within a wound or incision at the wound or incision site.

37. The decannulation detection system of claim 36, wherein the wound or incision comprises a stoma and the tube assembly comprises a tracheostomy tube assembly.

38. The decannulation detection system of claim 35, comprising an insulator positioned directly between each of one or more first capacitive plates and/or the one or more second capacitive plates to prevent direct contact between one or more first capacitive plates and the one or more second capacitive plates.

39. The decannulation detection system of claim 35, wherein, when the tube assembly moves away from the dressing during the decannulation event, the one or more first capacitive plates and the one or more second capacitive plates are moved away from each other by a corresponding distance, which causes a change in the capacitance measured between the one or more first capacitive plates and the one or more second capacitive plates.

40. The decannulation detection system of claim 35, wherein the tube assembly comprises a center portion and flanges, the flanges extending from opposite sides of the center portion.

41. The decannulation detection system of claim 40, wherein each flange comprises at least one of the one or more first capacitive plates, such that the tube assembly comprises a plurality of first capacitive plates and the dressing comprises a same quantity of the one or more second capacitive plates as a total quantity of plurality of first capacitive plates.

42. A method for detecting a decannulation event, the method comprising:
applying a dressing over a wound or incision site, the dressing comprising one or more first capacitive plates;
positioning the tube assembly in contact with the dressing, the tube assembly comprising one or more second capacitive plates;
providing an electrical circuit that is connected to the one or more first capacitive plates and the one or more second capacitive plates;
detecting, using a controller, a capacitance between the one or more first capacitive plates and the one or more second capacitive plates, wherein the capacitance varies based on a distance between the one or more first capacitive plates and the one or more second capacitive plates;
monitoring the capacitance; and
triggering, using the controller, an alert regarding a possible occurrence of the decannulation event when the controller detects that the capacitance between the one or more first capacitive plates and the one or more second capacitive plates is not within a predetermined range of capacitance values or is above or below a threshold capacitance value.

43. The method of claim 42, wherein at least a portion of the tube assembly extends at least partially within a wound or incision at the wound or incision site.

44. The method of claim 43, wherein the wound or incision comprises a stoma and the tube assembly comprises a tracheostomy tube assembly.

45. The method of claim 42, comprising positioning an insulator directly between each of one or more first capacitive plates and/or the one or more second capacitive plates to prevent direct contact between one or more first capacitive plates and the one or more second capacitive plates.

46. The method of claim 42, wherein, when the tube assembly moves away from the dressing during the decannulation event, the one or more first capacitive plates and the one or more second capacitive plates are moved away from each other by a corresponding distance, which causes a change in the capacitance measured between the one or more first capacitive plates and the one or more second capacitive plates.

47. The method of claim 42, wherein the tube assembly comprises a center portion and flanges, the flanges extending from opposite sides of the center portion.

48. The method of claim 47, wherein each flange comprises at least one of the one or more first capacitive plates, such that the tube assembly comprises a plurality of first capacitive plates and the dressing comprises a same quantity of the one or more second capacitive plates as a total quantity of plurality of first capacitive plates.

49. A decannulation detection system comprising:
a dressing for application over a wound or incision site, the dressing comprising one or more first inductors;
a tube assembly for positioning in contact with the dressing and comprising one or more second inductors;
an electric circuit that is connected to the one or more first inductors and the one or more second inductors; and
a controller that is configured to detect and monitor an inductance, optionally a mutual inductance, between the one or more first inductors and the one or more second inductors, wherein the inductance varies based on a distance between the one or more first inductors and the one or more second inductors;
wherein, when the controller detects that the inductance between the one or more first inductors and the one or more second inductors is not within a predetermined range of inductance values or is above or below a threshold inductance value, the controller is configured to trigger an alert regarding a possible occurrence of a decannulation event.

50. The decannulation detection system of claim 49, wherein at least a portion of the tube assembly extends at least partially within a wound or incision at the wound or incision site.

51. The decannulation detection system of claim 50, wherein the wound or incision comprises a stoma and the tube assembly comprises a tracheostomy tube assembly.

52. The decannulation detection system of claim 49, wherein:
the one or more first inductors each comprise coils of a wire in the electric circuit; and/or
the one or more second inductors each comprise coils of a wire in the electric circuit.

53. The decannulation detection system of claim 49, wherein, when the tube assembly moves away from the dressing during the decannulation event, the one or more first inductors and the one or more second inductors are moved away from each other by a corresponding distance, which causes a change in the inductance measured between the one or more first inductors and the one or more second inductors.

54. The decannulation detection system of claim 49, wherein the tube assembly comprises a center portion and flanges, the flanges extending from opposite sides of the center portion.

55. The decannulation detection system of claim 54, wherein each flange comprises at least one of the one or more first inductors, such that the tube assembly comprises a plurality of first inductors and the dressing comprises a same quantity of the one or more second inductors as a total quantity of plurality of first inductors.

56. A method for detecting a decannulation event, the method comprising:
applying a dressing over a wound or incision site, the dressing comprising one or more first inductors;
positioning the tube assembly in contact with the dressing, the tube assembly comprising one or more second inductors;
providing an electric circuit that is connected to the one or more first inductors and the one or more second inductors;
detecting, using a controller, an inductance, optionally a mutual inductance, between the one or more first inductors and the one or more second inductors, wherein the inductance varies based on a distance between the one or more first inductors and the one or more second inductors;
monitoring the inductance; and
triggering, using the controller, an alert regarding a possible occurrence of the decannulation event when the controller detects that the inductance between the one or more first inductors and the one or more second inductors is not within a predetermined range of inductance values or is above or below a threshold inductance value.

57. The method of claim 56, wherein at least a portion of the tube assembly extends at least partially within a wound or incision at the wound or incision site.

58. The method of claim 57, wherein the wound or incision comprises a stoma and the tube assembly comprises a tracheostomy tube assembly.

59. The method of claim 56, wherein:
the one or more first inductors each comprise coils of a wire in the electric circuit; and/or
the one or more second inductors each comprise coils of a wire in the electric circuit.

60. The method of claim 56, wherein, when the tube assembly moves away from the dressing during the decannulation event, the one or more first inductors and the one or more second inductors are moved away from each other by a corresponding distance, which causes a change in the inductance measured between the one or more first inductors and the one or more second inductors.

61. The method of claim 56, wherein the tube assembly comprises a center portion and flanges, the flanges extending from opposite sides of the center portion.

62. The method of claim 61, wherein each flange comprises at least one of the one or more first inductors, such that the tube assembly comprises a plurality of first inductors and the dressing comprises a same quantity of the one or more second inductors as a total quantity of plurality of first inductors.
